(19) United States
(12) Patent Application Publication
(10) Pub. No.: US 2026/0294619 A1
(43) Pub. Date: Oct. 1, 2026

(54) INTRAOCULAR LENSES AND METHODS AND SYSTEMS FOR INTRAOCULAR LENS IMPLANTATION

(71) Applicant: Apollo Medical, LLC, Dover, DE (US)

(72) Inventor: Manoj Motwani, San Diego, CA (US)

(21) Appl. No.: 19/367,220

(22) Filed: Oct. 23, 2025

Related U.S. Application Data
(63) Continuation-in-part of application No. 18/763,706, filed on Jul. 3, 2024.
(60) Provisional application No. 63/524,813, filed on Jul. 3, 2023.

(51) Int. Cl. A61F 2/16

(52) U.S. Cl. CPC A61F 2/1637; A61F 2/1662; A61F 2002/1681; A61F 2240/001

(57) ABSTRACT

Intraocular lenses are provided that include corrections for higher order aberrations. The intraocular lenses may include one or more positioning indicia on the lens or haptic portions. Methods of implanting intraocular lenses are provided comprising aligning laser positioning spots with the positioning indicia. Systems for implanting intraocular lenses comprise an eye tracker and a laser spot generator. Method of making intraocular lenses include incorporating higher order aberration corrections into a layer applied to a lens. The layer may include the positioning indicia.

### CROSS REFERENCE TO RELATED APPLICATIONS

This application claims priority to U.S. application Ser. No. 18/763,706 and U.S. application Ser. No. 18/763,717, both of which were filed on Jul. 3, 2024, and both of which claim priority to U.S. Provisional Application 63/524,813, filed on Jul. 3, 2023.

### BACKGROUND

A variety of methods and devices have been historically used to correct vision problems caused by various refractive anomalies of the eyes of a given subject. Several methods involve physical lenses placed over, on, or in the eye, such as external glasses or spectacles, contact lenses, and intraocular lenses. Refractive anomalies can also be corrected with selective laser ablation of the cornea.

An intraocular lens (IOL) is a small, artificial lens implanted in the eye during cataract surgery or refractive lens exchange to replace or augment the natural lens. It's designed to focus light onto the retina, restoring clear vision after the removal or augmentation of a cloudy or otherwise problematic natural lens. IOLs come in various types, including monofocal, multifocal, and toric lenses, each addressing different vision needs.

As initial background information regarding the physiology of the eye, several fundamental components of the human eye as shown in FIG. 1 include the cornea 12, the lens 14, and the retina 16. Between the cornea 12 and the lens is the iris 13. The anterior chamber 15 is between the posterior surface of the cornea 12 and the iris and the posterior chamber 17 is between the iris 13 and the lens 14. The optical axis 18 of the eye is an imaginary line passing approximately through the peak of the cornea and normal to the anterior corneal surface 12 straight back to a point on the retina 16. Ideally, light rays within a few millimeters of and parallel to the optical axis 18 that are incident to an eye will enter the cornea 12, pass through the lens 14 and will be focused essentially on a single point on the retina 16. The refractive power of the eye is typically measured in diopters and is the reciprocal of the focal length of the lens system formed by the cornea 12 and lens 14. Ideally, the power of the eye is such that the focal length is close to the axial length 20 of the eye. Typically, human eyes have a power of approximately 60 diopters, with about 40 diopters in the cornea and about 20 diopters in the lens.

Referring now to FIG. 2, for explanatory purposes, the cornea may be divided into an anterior portion 12a and a posterior portion 12b divided in FIG. 2 at the imaginary dotted line 12c running approximately through the middle of the stroma 34. The stroma 34 is the largest component of the cornea. The stroma 34 is a layered cellular structure that may be about 450-500 micrometers thick for a normal cornea and comprise around 90% of the total corneal thickness. The most anterior component of the anterior portion 12a of the cornea illustrated in FIG. 2 is the epithelium 32, which comprises several layers of cells that total typically about 50-60 micrometers thickness and are about 10% of the total corneal thickness. Although not shown in FIG. 2, there is a thin layer of water called the tear film that coats and lubricates the outer air exposed surface of the epithelium 32. The other layers of the cornea denoted 33, 35, and 36 in FIG. 3 are quite thin relative to the stroma 34 and the epithelium 32 although they can be significant functionally and clinically. The most posterior component of the posterior portion 12b of the cornea is the endothelium 36, which is a cell monolayer a few micrometers thick. The cornea further comprises thin acellular layers 33 and 35 at the boundaries between the stroma cells and the epithelium 32 and the endothelium 36 respectively, typically comprising collagen as well as other biological molecules. Although illustrated as single membranes 33 and 35 in FIG. 2, these layers have recognized sub-layers with different components and characteristics. The layer 33 between the epithelium and the stroma may comprise the epithelium basement membrane and Bowman's membrane. The layer 35 between the endothelium and the stroma may comprise Dua's layer and Descemet's membrane.

Common refractive errors of the eye include myopia, hyperopia, and astigmatism. With myopia, the anterior corneal surface curvature may be substantially spherical, but the curvature and hence the power is too large. With hyperopia, the anterior corneal surface curvature may also be substantially spherical, but the curvature and hence the power is too small. With astigmatism (also referred to as cylinder), the curvature of the anterior corneal surface and/or the lens 14 may have a non-spherical football type contour, with a different radius of curvature in different directions from the peak. For example, the anterior corneal surface could have a maximum power along a first circumferential meridian and a minimum power in a circumferential meridian approximately orthogonal to that. Although there are different ways to numerically represent it, a characterization of an astigmatic eye may include two values, one related to the maximum and minimum refractive powers in the different directions (e.g., the difference between these two powers) and an angle defining the deviation from horizontal of the lower power meridian.

Referring now to FIG. 3, refractive aberrations of an eye may be characterized by the coefficients of polynomials called Zernike polynomials (although other representations such as Fourier series can also be used). Myopia, hyperopia, and lower order astigmatism are characterized by coefficients of second order Zernike polynomials denoted 24 in FIG. 3. These are lower order aberrations (LOA). Optical aberrations of the eye can also include aberrations characterized by coefficients of third and higher order Zernike polynomials and may be referred to as higher order aberrations (HOA) denoted as 26 in FIG. 3. High order aberrations characterized by Zernike polynomials include aberrations known as coma, trefoil, quadrafoil, and spherical aberration. As can be seen in FIG. 3, there is also higher order astigmatism known as “secondary” astigmatism that may be present, but when the term astigmatism is used alone, it is lower order astigmatism that is being referred to. The refractive properties of any given eye as a whole may be expressed as a combination of one or more of these Zernicke characterized refractive aberrations in varying amounts. Also, the surface contour of the anterior portion of the eye can also be expressed as a combination of one or more of these Zernicke polynomials.

While traditional refractive errors such as myopia, hyperopia, and astigmatism are well understood and routinely corrected by glasses or conventional IOLs, a more complex class of visual imperfections, known as higher-order aberrations (HOAs), remains a significant clinical challenge both from a measurement standpoint and logistics standpoint. These aberrations are caused by subtle and unique irregularities in the shape of the cornea or the eye's crystalline lens, creating a highly specific distortion of light as it passes through the optical system. While most people have subclinical HOAs that do not significantly impact their vision, a substantial patient population, particularly those in need of cataract surgery and others with corneal irregularities like keratoconus or those who have undergone prior refractive surgeries like LASIK, can experience visually significant and debilitating symptoms.

The manifestations of HOAs go beyond simple blurriness and include a host of qualitative visual disturbances that profoundly affect a patient's quality of life. These include ghost images, halos, starbursts, and glare, especially under mesopic and scotopic conditions (i.e., low-light environments) when the pupil dilates and exposes a wider, more irregular optical surface. There are over 60 different types of HOAs, with the most disruptive being vertical and horizontal coma, trefoil, and spherical aberrations, each of which causes light to spread out in a distinct pattern. The highly specific nature of these symptoms makes diagnosis and effective treatment difficult with traditional methods. The market for a solution is not merely for cosmetic improvement but for a fundamental restoration of functional vision, particularly for activities like night driving.

As shown in FIG. 4, an examination of a patient with, for example, a phoropter 44 may be performed to generate a “manifest” refraction measurement to generate a correction prescription 46. With a phoropter 44, by looking through a sequence of different lenses and determining which corrections look best to the patient in terms of perceived visual quality, sphere and cylinder corrections are found that compensate for the patient's myopia, hyperopia, and/or astigmatism. These measurements may be used to generate a prescription 46 for glasses, contact lenses, or intraocular lenses. In some cases, the manifest refraction prescription 46 may be used as illustrated by block 48 to generate a lens contour specification for glasses, contact lenses, or intraocular lenses or a treatment map defining how to re-shape the cornea 12 to generate phoropter determined sphere and cylinder corrections. A phoropter, however, cannot be used to measure higher order aberrations.

The laser ablation procedures that have been developed have attempted to correct untreated refractive aberrations of the eye by altering the anterior shape of the cornea to remove or otherwise counteract the refractive aberrations of the untreated eye. Fundamentally, portions of the cornea are ablated away to change the shape of the air/cornea interface in a manner that reverses the refractive aberrations of the untreated eye. In this context, a treatment map defines stromal tissue removal depths at different locations on the anterior corneal surface to generate a desired cornea curvature after the laser ablation surgical procedure is complete.

Although limited attempts to correct HOAs with glasses or IOLs have been introduced, laser ablation procedures have been more extensively used conventionally to treat them and accordingly some existing HOA treatment methodologies are presented below in this context. Laser ablation procedures perform ablation of stromal tissue 34 and come in two basic types that differ in how the stroma is exposed to the laser for the ablation procedure itself. In photorefractive keratectomy (PRK), the epithelium 32 over the portion of the stroma 34 to be treated is removed to expose the stromal tissue for laser ablation. This removal may be done mechanically with a brush for example, chemically, with laser pulses, or possibly a combination of such techniques. The underlying stromal tissue is then ablated to change the shape of the cornea as desired. After the procedure, the epithelium grows back over the stromal tissue in a few days or weeks after the surgery. With laser assisted in-situ keratomileusis (LASIK), a flap is created which may be about 100 micrometers deep through the epithelium and the outermost portion of stromal tissue with a mechanical blade or a laser. This flap is pulled back, exposing anterior stromal tissue which is then ablated to change the shape of the cornea as desired. After the stromal ablation, the flap is folded back over the surgical site where it heals back in place. Typically, LASIK has a shorter recovery period and less post-surgical discomfort than PRK. In both cases, stromal tissue is selectively removed by laser ablation to correct refractive errors. All the methods and apparatus described herein are equally applicable to either or both PRK, LASIK, or other to be developed surgical techniques involving modifying stromal tissue to improve vision.

As an alternative and/or supplement to the manifest refractive measurements of FIG. 4, more automated and objective methods and systems have been developed to characterize the refractive properties and shape characteristics of the eye. For example, a technique known as wavefront guided laser surgery has been developed. Referring to FIG. 5, an instrument called a wavefront aberrometer 52 is used to characterize the properties of the patient's eye. These instruments, such as a Hartmann-Shack wavefront aberrometer, may shine a spot of light onto the retina, and the retina reflected wavefront that exits the eye is detected through a lens array. As the reflected light travels out of the eye, the wavefront exiting through the pupil opening is distorted, and detecting these distortions can characterize the total optical aberrations 53 introduced into the light beams by the optical elements and interfaces of the patient's eye.

Another modality shown in FIG. 6 was introduced in the field of laser eye surgery and is known as topographic guided ablation, which provides more enhanced imagery of the anterior corneal surface thereby providing the ability to more effectively map and define HOAs as irregularities in the surface elevation of the anterior corneal surface. Although topographic guided ablation was initially used for repair of damaged, diseased, or otherwise abnormal corneas, it is also used for primary refractive correction in laser eye surgery on patients with otherwise normal corneas.

Referring now to FIG. 6, for topographic guided ablation, rather than using a wavefront aberrometer 52, a corneal topology measuring instrument 56 is utilized. Corneal topology measuring instruments 56 are configured to generate high resolution elevation measurements 55 of the anterior surface of the cornea. Such maps can be very high resolution and may map the elevation of many thousands of points in a corneal area of a few millimeters diameter. These devices detect light reflected from the tear film, anterior epithelium surface, and/or epithelium basement membrane. The WaveLight Topolyzer VARIO from Alcon is one example of a commercially available corneal topology measuring instrument 56 that uses a Placido ring illumination and analysis technique. Another method of acquiring corneal topology is known as a Scheimpflug imaging system. This technique acquires images of slices of the eye at different angles. A Scheimpflug camera may acquire a variety of structural information regarding the anterior and posterior surfaces of the cornea with these slice images. This information may include high resolution elevation data for the anterior corneal surface.

A fundamental difference between wavefront aberrometry and corneal topography is that wavefront aberrometry provides information on the combined optical properties of the eye as a whole (e.g. including the anterior epithelium/corneal region, the posterior occular region, the lens, the retina, etc.), whereas corneal topography provides information only regarding the surface shape of the anterior surface of the eye without measuring the optical characteristics of other optical elements of the eye. Topographic measurements were not used to correct astigmatism until the results of the Inventor's clinical studies were completed in 2016. Instead, topographic-guided ablation systems relied on manifest refraction measurements for input to treat astigmatism, which is an essentially lower order aberration. Since topographic measurements of lower order astigmatism often differed from manifest measurements, established science theorized that the differences were due primarily to posterior ocular irregularities; therefore, requiring the use of manifest measurements for correction of astigmatism whenever topographic measurements differed from manifest.

Topographic-guided ablation systems have now been significantly improved based on the new science established from the Inventor's studies, which provided new systems and methodologies for the use of topographic measurements to not only ablate HOAs, but to reduce astigmatism in accordance with anterior corneal astigmatism based at least in part on topographic measurements.

Since PRK and LASIK were introduced, the aim has been to achieve the anterior corneal shape from an ablation pattern derived from devices and methods that have an established medical and scientific basis for providing the best possible optical quality. Although this is the aim, the systems, methods and reasons to achieve such anterior corneal shape vary and those variances relate as much to differing scientific views as to available technology. A large amount of research has been performed by surgeons in the field to attempt to define the best way to treat various refractive conditions of the eye to produce the best outcomes for subjects of these procedures. Such research continues to be performed as available measurement technologies have become available.

Commercialized corneal laser ablation systems and methods have been based on the premise that the ablation pattern configured for a subject, such as for astigmatism, should be derived from a quantification of optical quality that is determined using subjective and/or objective data derived from “whole” eye measurements, including anterior and posterior regions. The reasoning for this premise is based on established medical and scientific postulation that astigmatism is caused by refractive anomalies that are present throughout a subject's entire ocular focusing system, including the posterior ocular region. Under this premise, in order to achieve the best possible optical quality (based on established medical and scientific principles) an ablation pattern for correcting astigmatism had been and was largely medically and scientifically required to be derived from “whole” eye measurements prior to the Inventor's studies in 2016.

The Inventor challenged such established medical and scientific principles and developed systems and methods for corneal laser ablation that are novel and contrary to certain established medical and scientific principles. It was the Inventor's premise that the ablation pattern for a subject with astigmatism should not be derived solely from manifest measurements, but rather from a quantification of anterior surface features which are necessary to create a more uniform cornea to make the stromal surface as smooth and spherical as possible.

Results from the Inventor's early clinical studies found that the primary reason for the differences between topographic measurements and manifest measurements was not posterior ocular irregularities as postulated by medical and scientific theory, but rather the effects of anterior corneal HOAs on such measurements. The clinical studies showed that if HOAs of a subject were not first ablated in accordance with topographic measurements, manifest measurements of that subject generally showed false astigmatism measurements due to the masking effect of the HOAs. The more significant the anterior corneal HOAs, generally the more significant the false astigmatism measurements. As a result of the studies, the Inventor created the LYRA™ (i.e. Layer Yoked Reduction of Astigmatism) protocol, which is a medical methodology developed by the Inventor which used corneal astigmatism based on topographical measurements, as a better methodology for programming of ablation patterns to correct astigmatism. Systems and methods such as those described in the Inventor's U.S. Pat. No. 10,857,032 (incorporated herein by reference in its entirety) are modalities that utilize these principles.

Another significant variable in the ocular focusing system is the epithelial layer of the cornea, which is the outermost or most anterior layer. This epithelial layer is the only corneal layer that regenerates; the underlying stromal layer (the “meat” of the cornea where laser ablation is performed) and the endothelium (the innermost layer which pumps fluid out of the cornea to keep it optically clear) do not regenerate. But, since the epithelial layer does regenerate, such layer can significantly vary based on a number of factors, including HOAs, disease or trauma. However, although the epithelium may vary, it is not considered to be variable in the normal Gullstrand models of the eye, which are used for existing topographic or wavefront mapping. As a result, existing modalities fail to properly measure the epithelial thickness over the anterior cornea following the shape of the cornea underneath. The Inventor's studies have shown that the epithelium varies in thickness rather dramatically. It thickens to fill in flatter “low” stromal areas and thins over steeper “high” stromal areas (referred to as “epithelial compensation”). This has the effect of masking some of the anterior stromal corneal irregularities, and thus creating topography and wavefront mapping that would not properly measure the actual corneal irregularity.

Over the decade prior to 2017 some experimental devices were used by surgeons to measure epithelial thickness and document epithelial compensation. Furthermore, attempts have been made to map the epithelial basement membrane, which would map the anterior epithelium-stromal border directly. In late 2016 and 2017 the Inventor realized that the epithelium had to be playing a part in compensation for corneal irregularities as he noticed evidence of change on topographic maps in the days and weeks after laser treatment with the LYRA™ Protocol. In 2017, Optovue introduced the first commercial epithelial thickness mapping devices and in September 2017 the Inventor obtained one for use and began correlating epithelial compensation with anterior corneal irregularities. He published this information in a paper in 2020 describing reasons for inaccuracy of outcomes after treatment with Contoura with LYRA ™ Protocol for primary LASIK corrections, affecting a percentage of primary LASIK corrections. This led the inventor to create a system, methodology and device to measure and treat epithelial compensation of corneal irregularity along with topographic guided ablation to make a more uniform cornea. Systems and methods such as those described in the Inventor's U.S. Pat. No. 10,857,033 (incorporated herein by reference in its entirety) are modalities that utilize these principles.

Further research led to the discovery of high levels of epithelial compensation, with 30 plus microns of epithelial compensation in certain situations; thereby significantly masking stromal irregularity. The Inventor then developed a new medical procedure based on his clinical studies involving epithelial compensation, the Corneal Repair Epithelium Adjusted Topography Enhanced Protocol (CREATE™ Protocol) to address this medical condition. This protocol treats the stroma masked by the epithelial compensation and then utilizes topographic guided ablation using the LYRA™ Protocol to increase higher order aberration reduction and create as uniform, regular cornea as possible.

Using the CREATE protocol, the Inventor began to treat this masking by performing surface excimer laser ablation (phototherapeutic keratectomy or PTK) to the central anterior epithelium to the depth determined by the thickest area in the central 5 mm optical zone. As epithelial tissue and stromal tissue are removed similarly by excimer laser, this effectively removed anterior stromal irregularity that could not be measured otherwise due to the epithelium masking. This was followed by topographic guided ablation that removed the remainder of irregularity measurable by topography.

The results from the Inventor's clinical studies using the new CREATE™ Protocol in a newly configured methodology which included epithelial compensation factors on subjects diagnosed with keratoconous, an eye condition in which the cornea gets thinner and gradually bulges outward into a cone shape, showed an average HOA reduction of 57% (in contrast with prior methods getting only about 31% reduction), and a dramatic decrease in a subject's optical aberration. HOA reduction may have improved even more with the CREATE ™ Protocol if the Inventor didn't limit the corrections performed to the astigmatism correction limitation of the Contoura laser system. The corresponding increase in vision significantly improves the outcomes for treatment of keratoconus over prior procedures. The beneficial results from this breakthrough clinical procedure for keratoconus by the Inventor have been submitted for publication to the medical community and were an inspiration for the Inventor in creating the novel systems and methodologies embodied herein.

Another technique that has been experimented with over the past decade is a system referred to as ray tracing. Ray tracing is a computer-based method used to calculate an ablation profile for a refractive laser by incorporating data derived from several types of measurements. It may incorporate a combination of information such as wavefront guidance to determine theoretical posterior ocular aberrations as well as axial length of the cornea and topography information to determine the curvature of the cornea at each point measured, and therefore the corneal power and astigmatism as well. It attempts to treat all of these per point by creating a ray tracing/vector diagram for each point and therefore treat, theoretically, all sources of aberrations through the entire ocular focusing system. However, based on the FDA study submitted with its approval in 2025, the system, similarly to all wavefront-based systems, has the major deficiency of increasing HOA, not decreasing HOA. Further, it has other deficiencies, such as not effectively accounting for epithelial compensation. Most of these deficiencies may be overcome by embodiments provided herein.

It should be noted that this Background is not intended to be an aid in determining the scope of the claimed subject matter nor be viewed as limiting the claimed subject matter to implementations that solve any or all the disadvantages or problems presented above. The discussion of any technology, documents, or references in this Background section should not be interpreted as an admission that the material described is prior art to any of the subject matter claimed herein.

### SUMMARY

Embodiments of this disclosure include corneal laser ablation systems configured and programmed to perform the methodologies derived from the disclosures herein, some of which are provided in separate patent applications related to U.S. Provisional Application 63/524,813, filed on Jul. 3, 2023. The summary of embodiments below is directed to intraocular lenses and intraocular lens systems configured and programmed to perform the methodologies derived from some of the disclosed systems of the disclosures herein, not to all of the systems and methods that are possible from the disclosures herein.

In some embodiments, an intraocular lens comprises an optic portion and a haptic portion. and one or more positioning indicia. The optic portion comprises corrections for higher order aberrations, wherein the corrections are derived at least in part from an aberration map of an anterior corneal surface of a subject (the “custom aberration map”), and wherein the corrections comprise corrections for one or more 3^{rd} or 4^{th} order corneal higher order aberrations selected from trefoil, coma, quadrafoil, and higher order astigmatism that are present in the custom aberration map.

In some embodiments, a method of making an intraocular lens comprises creating an aberration map of an anterior cornea of a subject (the “custom aberration map”) and deriving aberration corrections from the custom aberration map, wherein the corrections comprise one or more 3rd or 4th order corneal higher order aberrations selected from trefoil, coma, quadrafoil, and higher order astigmatism that are present in the custom aberration map. The method further comprises forming an intraocular lens containing the aberration corrections.

In some embodiments, an intraocular lens comprises an optic portion, and a haptic portion, and the optic portion comprises corrections for higher order aberrations. The corrections for HOA may be derived from corneal topography data. The corrections for HOA are incorporated into a coating layer applied to the optic portion.

In some embodiments, a method of implanting an intraocular lens comprises inserting an intraocular lens under a cornea of a subject, directing a light beam onto a defined location on the cornea, and aligning a defined portion of the intraocular lens with the defined location. The method may comprise tracking the position of the subject's eye during the aligning. The intraocular lens being implanted may comprise a positioning indicia. The intraocular lens being implanted may comprise corrections for HOA.

In some embodiments, a system for surgically implanting an intraocular lens comprises a surgical viewer (generally, a viewing microscope when performed by a surgeon, but, if done robotically, the viewer could be any optical viewing device, including cameras), an eye tracker, and a laser that is coordinated to the eye tracker and configured to inject a laser beam into an intraoperative optical viewing path to create a laser spot on the cornea while implanting the intraocular lens. The laser may be configured to receive information regarding a desired location of a portion of an intraocular lens with respect to a cornea of a surgical subject. The location of a portion of an intraocular lens may comprise a positioning indicia marked on the intraocular lens.

The system for surgically implanting an intraocular lens can be performed by a surgeon or it can be performed by an automated device such as a robotic arm, wherein the device is configured to include mechanisms that can automate each of the functions described for the system and implant the intraocular lens.

In some embodiments, a method of making an intraocular lens is provided. The method may include acquiring measurements of one or more lower order whole eye optical aberration characteristics at least in part via retinal reflection measurements and deriving therefrom a first astigmatism characteristic representative of a lower order whole eye astigmatism. The method may further comprise acquiring measurements of one or more lower order elevation characteristics of the anterior corneal surface of the eye at least in part from one or more of either tear film, anterior epithelium, and/or epithelium basement membrane reflection measurements and deriving therefrom a second astigmatism characteristic representative of a lower order anterior corneal surface astigmatism. The first astigmatism characteristic may be compared with the second astigmatism characteristic to generate an estimate of an amount of difference between lower order whole eye astigmatism and lower order anterior corneal surface astigmatism. Based at least in part on the comparing, a third astigmatism characteristic is determined for inclusion in the generation of the treatment map. The third astigmatism characteristic is determined using either (1) one or both of the first astigmatism characteristic and the second astigmatism characteristic or (2) a derivative of one or both of the first astigmatism characteristic and the second astigmatism characteristic. The method further includes incorporating a vision correction based at least in part on the third astigmatism characteristic into the optic portion of the intraocular lens.

In some embodiments, a system for surgically implanting an intraocular lens into an eye of a subject comprises a surgical viewer and a wavefront aberrometer. The wavefront aberrometer comprises a display configured to display optical aberrations of the eye of the subject during the surgical procedure while the intraocular lens is manipulated during and/or after the implanting.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

Various embodiments are discussed in detail in conjunction with the Figures described below, with an emphasis on highlighting the advantageous features. These embodiments are for illustrative purposes only and any scale that may be illustrated therein does not limit the scope of the technology disclosed. These drawings include the following figures, in which like numerals indicate like parts.

FIG. 1 is a cross section of eye anatomy.

FIG. 2 is a close-up view of the cornea of FIG. 1.

FIG. 3 illustrates low order and high order aberrations.

FIG. 4 is a prior art method for treatment map generation using a phoropter.

FIG. 5 is a prior art method for treatment map generation using a wavefront aberrometer.

FIG. 6 is a prior art method for treatment map generation using a corneal topography measuring instrument.

FIG. 7 is a treatment map generation system in accordance with some embodiments of the invention.

FIG. 8 illustrates using a treatment map in a corneal laser ablation system.

FIGS. 9A, 9B, and 9C illustrate a hardware configuration that may be suitable for implementing the systems in accordance with FIG. 8.

FIG. 9D illustrates another hardware configuration that may be suitable for implementing the systems in accordance with the principles of FIG. 8.

FIG. 10A is an example user-interface that may be associated with the system of FIG. 8.

FIG. 10B is another example user-interface that may be associated with the system of FIG. 8.

FIG. 11 is a flowchart of a method of corneal laser ablation in accordance with some embodiments of the invention.

FIG. 12 is a flowchart of another method of corneal laser ablation in accordance with some embodiments of the invention.

FIG. 13 is a flowchart of another method of corneal laser ablation in accordance with some embodiments of the invention.

FIGS. 14A and 14B illustrate using a lens contour specification in a method of making lenses for glasses or contact lenses.

FIG. 15 shows several embodiments of intraocular lenses.

FIG. 16 shows an intraocular lens implanted in an eye.

FIG. 17 is a block diagram of a system for intraocular lens implantation.

FIG. 18A is a block diagram of another system for intraocular lens implantation.

FIGS. 18B and 18C illustrate the use of the system of FIG. 18A to implant an intraocular lens.

FIG. 19 shows another implantation of an intraocular lens using the system of FIG. 18A.

FIG. 20 shows another implantation of an intraocular lens using the system of FIG. 18A.

FIGS. 21A and 21B illustrate using a lens contour specification in a method of making intraocular lenses.

FIG. 22 is a flowchart of another method of corneal laser ablation in accordance with some embodiments of the invention.

### DETAILED DESCRIPTION

The following description and examples illustrate some exemplary implementations, embodiments, and arrangements of numerous inventions, including the invention for specialty intraocular lenses and methods and systems for intraocular lens implantation, some of which relate to U.S. Provisional Application 63/524,813, filed on Jul. 3, 2023. Those of skill in the art will recognize that there are numerous variations and modifications of these inventions that are encompassed by its scope. Accordingly, the description of a certain example embodiment should not be deemed to limit the scope of the inventions.

To facilitate an understanding of the various embodiments described herein, a number of terms are defined below.

PRK: An acronym for Photorefractive Keratectomy. PRK is a laser ablation procedure wherein the epithelium covering the corneal stromal tissue is removed mechanically, chemically, and/or with laser ablation as part of a laser ablation refractive correction treatment on an eye. Following PRK, the epithelium grows back over the corneal stromal tissue that has been modified with laser ablation for refractive corrections.

LASIK: An acronym for Laser-Assisted In Situ Keratomileusis. LASIK is a laser ablation procedure wherein a hinged circular or approximately circular flap of typically about 100 to 160 micrometer thickness and 8 to 10 mm diameter is pulled off of the anterior portion of the cornea to expose a surface of corneal stromal tissue to which laser ablation for refractive correction is applied. The perimeter of the flap may be cut mechanically with a blade or may be cut with a laser. The flap thickness typically spans the epithelium, the basement membrane, and an anterior portion of the corneal stromal tissue. After the laser ablation for refractive correction is performed, the hinged flap is placed back over the treated corneal tissue. With LASIK, the epithelium remains substantially intact, and the post treatment healing occurs around the perimeter of the flap and at the corneal tissue interface underneath the flap.

Anterior Ocular Region: The anterior cornea that includes the tear film, the epithelium, the basement membrane, and corneal stroma.

Posterior Ocular Region: All structures of the eye beneath (posterior to) the anterior ocular region. These include, for example, the posterior surface of the cornea and the lens.

Astigmatism: A shape characteristic and/or optical refractive characteristic of one or more surfaces of an optical system that correspond to the Zernicke polynomials of radial order 2 and angular order +2 and −2. An optical system may have multiple refractive surfaces each of which has an individual astigmatism shape characteristic. The astigmatic shape characteristics of the individual surfaces will combine to generate a total optical refractive astigmatism characteristic of the optical system as a whole. The contribution that each refractive surface makes to the total astigmatism of the optical system will depend on the astigmatic shape characteristic of the surface and the index of refraction change occurring across that surface. For an eye, surfaces along the optical path of the eye that may have an astigmatism shape characteristic include the anterior corneal surface, the posterior corneal surface, the anterior surface of the lens, the posterior surface of the lens, and the retina. The term astigmatism characteristic or astigmatic characteristic may be used herein to refer to a topographical shape of a single surface or to an optical refraction characteristic of either a single surface or a collection of surfaces forming all or part of an optical system. When the term astigmatism or astigmatic is applied to an optical system or part thereof with more than one refractive surface it refers to an optical refraction characteristic of the combination of refractive surfaces that make up the optical system or part thereof. When the term astigmatism characteristic or astigmatic characteristic is applied to an individual surface it refers to a topographical shape characteristic of the individual surface. Astigmatism measured by a topographic measuring technology refers to an astigmatic physical contour shape characteristic of the surface measured. Astigmatism measured by wavefront aberrometry refers to the total astigmatic optical refractive aberration generated as the wavefront travels through the one or more surfaces during the measurement. As used herein, refractive astigmatism refers to an astigmatic optical refractive property of a surface or collection of surfaces and topographic astigmatism or shape astigmatism refers to an astigmatic physical contour of a single surface of an optical system. These are not necessarily identical but are related because refractive astigmatism of an optical system or part thereof is produced by topographic astigmatism of the surface or surfaces making up the optical system. In some cases, the term astigmatism as used herein is explicitly applied to the topographic shape of a surface. In some cases, the term astigmatism as used herein is explicitly applied to the refractive properties of all or part of the eye. In some cases, the term astigmatism is used alone in which case it may refer to either or both depending on the context.

Posterior Ocular Astigmatism: Astigmatic refractive aberrations caused by astigmatic shape characteristics of the eye in the posterior ocular region.

Anterior Corneal Astigmatism: Astigmatic topographic shape characteristics of the anterior surface of the cornea.

Posterior Corneal Astigmatism: Astigmatic topographic shape characteristics of the posterior surface of the cornea.

Lenticular Astigmatism: Astigmatic refractive aberrations of the lens as a whole.

Retinal Astigmatism: Astigmatic topographic shape characteristics of the retina.

Whole Eye Astigmatism: Astigmatic refractive aberrations caused by the combination of astigmatic shape characteristics present throughout the entire eye focusing system.

Manifest Astigmatism: The astigmatic refractive aberrations perceived by a subject. The manifest astigmatism may or may not be the same as the whole eye astigmatism because the visual perception of the subject may be further influenced by shape and or refractive higher order aberrations also present in some or all of the eye.

Epithelial Compensation: Epithelial growth that is reduced or increased due to aberrations in the anterior ocular region that results in differences in epithelial thickness over a defined area of the anterior ocular region. Numerically, epithelial compensation can be quantified as the thickness difference between the thickest part of the epithelium and the thinnest part of the epithelium in a defined region of the anterior surface of the eye, for example in a region of defined diameter centered on the corneal apex.

Lower Order Aberrations (LOA): Shape and/or refractive characteristics of the eye or one or more parts of the eye that correspond to Zernicke polynomials of radial order 2 or less. As described above with respect to the term astigmatism, lower order aberrations may, depending on the context, refer to the topographic shape characteristics of an individual refractive surface or may refer to the optical refractive aberrations of an optical system or portion thereof with more than one refractive surface.

Higher Order Aberrations (HOA): Shape and/or refractive characteristics of the eye or one or more parts of the eye that correspond to Zernicke polynomials of radial order 3 or more. As described above with respect to the term astigmatism, higher order aberrations may, depending on the context, refer to the topographic shape characteristics of an individual refractive surface or may refer to the optical refractive aberrations of an optical system or portion thereof with more than one refractive surface.

Based on clinical studies performed by the Inventor over many years, the Inventor had previously concluded that in order to achieve the best optical quality for most subjects, a corneal laser ablation system should be configured with the particular devices and data input necessary to derive a laser ablation pattern calculated to create the most uniform cornea possible. The systems and methods that can be created from the information and embodiments herein further expand on the Inventor's challenge of established medical and scientific principles that it is the uniformity of the cornea, not the subjective and/or objective data of whole eye measurements, that is the key to creating the best possible optical quality for a subject. Systems and methods described herein expand the applicability of the treatments developed by the Inventor to additional patients and provide methods and systems for corneal laser ablation treatments that are more easily used and flexible for surgeons to adapt to the wide variety of patients presented to them for treatment.

Based on those clinical studies, the Inventor expanded the potential applications beyond the basic novel systems for corneal laser ablation and developed novel systems and methods for creating and implanting interocular lenses. An important element of these novel systems and methods is the ability to create an IOL that can reduce HOA. Correcting HOA had previously been problematic. Until the Inventor validated the new science associated with the Inventor's corneal laser ablation systems and methods, it had not been practical to reduce HOA via IOLs. The new science developed by the Inventor has enabled this new system and methodology for IOLs that can dramatically improve eyesight for patients undergoing cataract surgery and lens replacement or augmentation.

Elements of a basic novel system in accordance with embodiments described herein may include:

Topography imaging (e.g. Placido or Scheimpflug imaging) that uses a best fit sphere or other software technology to image elevations and depressions in the anterior corneal surface to create a map of the lower order and higher order aberrations of the anterior corneal surface. This will provide the lower order aberrations in the form of topographic astigmatism of the anterior cornea in (for example) magnitude of diopters and the axis of the topographic astigmatism once the higher order aberrations are removed.

Wavefront imaging utilizing an aberrometer such as Hartmann-Schack. Alternatively or in combination, an automated or manual phoropter may be used. This will provide whole eye higher order refractive aberrations as well as whole eye lower order refractive aberrations such as a magnitude and axis of refractive lower order astigmatism. The wavefront aberration map will include all sources of refractive lower order astigmatism including refractive posterior ocular astigmatism that can be caused from posterior cornea, crystalline lens, the retina, or any other source of refractive posterior ocular astigmatism. This data may be important to be examined as it contains one of the two variable HOA/LOA sources in the eye, the crystalline lens.

Epitheilal compensation data of anterior stromal irregularity cannot be measured by the above two sources and constitutes the second variable HOA source in the eye. Epithelial compensation occurs when the epithelium thins over stromal irregularity elevations and thickens over stromal irregularity depressions. This can be measured either by OCT mapping of epithelial thickness, or by technology mapping the edge of the stroma/basement membrane. This interface can be mapped with certain technologies as well. This epithelial compensation map can be matched to the anterior corneal elevation map from topography.

Measurements from an interferometer can provide axial length which can be used with the corneal topography information to provide lower order spherical power information.

A processor configured to use an algorithm to combine data from the wavefront aberrometer, the corneal topography measuring device, and/or the epithelial thickness measuring device into a treatment map for a corneal laser ablation treatment A tracking system that uses a CCD camera to image corneal and iris features for each one of these ocular imaging technologies to align the maps appropriately.

A software system that uses comparative technology and/or artificial intelligence technology to align the imaging maps utilizing any or all of the following: the higher order aberration maps from topographic imaging and wavefront, the lower order aberration maps from the topography imaging and the wavefront imaging (and/or manifest), and the elevation/depression map from the anterior corneal imaging and the epithelial compensation.

A software rejection technology that rejects the combined map if too many disparities are present, or rejects the wavefront map if the anterior topography map and wavefront map show similar HOA and/or lower order astigmatism magnitude and axis, and if HOA maps demonstrate senile lens changes.

An axial length auto refraction technology that utilizes the wavefront imaging to provide a refraction of the eye that provides spherical power information.

A final software imaging map that incorporates any or all: HOA, LOA including astigmatism magnitude and sphere, spherical aberration and spherical power. This information can be transferred to an excimer laser system for laser correction of HOA and LOA to create an ocular system that minimizes HOA and LOA while eliminating variables from epithelial compensation or posterior ocular astigmatism sources.

The Inventor's findings concerning epithelial compensation of HOA/corneal irregularity has proved to be highly important for not only corneal laser repair of past surgery, disease, trauma, etc, but also for treating and/or preventing inaccurate outcomes of primary virgin eye LASIK and PRK procedures. As a result, the Inventor has configured new systems and methodologies using epithelial compensation principles along with the Inventor's LYRA™ and CREATE ™ Protocols to significantly enhance corneal laser ablation and improve resulting outcomes. An advantageous feature of the embodiments of the new systems and methodologies with respect to corneal laser ablation is that it creates an ablation pattern that shapes the stromal surface that the epithelium grows over as smooth and spherical as possible, minimizing post-surgical epithelium issues. The configurations embody essentially three separate systems that can deliver data using a processing methodology that can be incorporated into a comprehensive laser ablation treatment map to allow for better corneal uniformity, elimination of most or all ocular focusing aberrations, and prevention of further epithelial compensation. An embodiment combines these three technologies, more fully described herein, to create a final HOA and LOA map for a particular eye. However, because IOLs generally do not involved corneal laser ablation, the epithelial compensation element may be omitted for IOL systems.

In another embodiment the data in creating the final HOA and LOA map is subjected to comparative analysis to eliminate and/or require rescan of aberrant data that could include data from operator error.

Inventive embodiments described herein may utilize three different technologies to measure HOAs and LOAs of the cornea. The first technology is topography mapping, which can be from either Placido or Scheimpflug imaging. The second technology is wavefront imaging, which maps whole eye HOA and LOA and axial length, such as from Hartmann-Shack. The third technology is epithelial mapping measurement by either OCT or other technology. Certain embodiments herein include combining the data from these different imaging technologies to create a comprehensive map for treatment of all HOAs and LOAs, including the HOA masked by epithelial compensation. This system is designed to minimize all HOA and LOA in the ocular focusing system to create as close to a virtually perfect ocular focusing system as possible by treating the combined data on the cornea with an excimer laser system to make a more uniform cornea.

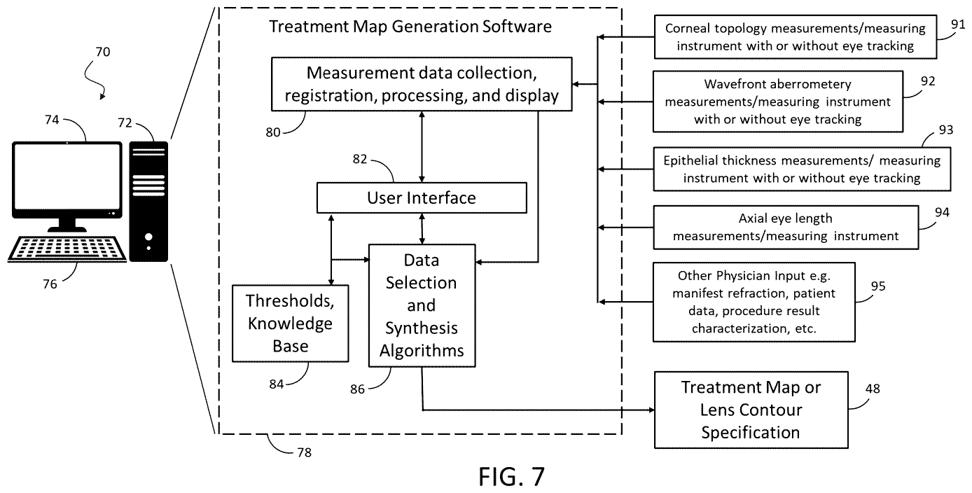

An example of a system for implementing the above principles is shown in FIG. 7. Referring now to this Figure, the system includes a general-purpose computer system 70 which hosts or executes, directly or indirectly, treatment map or lens contour specification generation software 78 described in more detail below. The treatment map or lens contour specification generation software 78 may reside on and be executed entirely by/on a local computer processing system 72 located at, for example, an optometrist, ophthalmologist, or LASIK surgeon's office or clinic. Alternatively, the treatment map or lens contour generation software may be hosted on the Internet and accessed, for example, with a browser or other software that is executed on the processing system 72. The generation software 78 may comprise processor executable instructions stored in a computer readable memory wherein the instructions are configured to cause a general purpose computer to perform the acts described herein for treatment map or lens contour specification generation. As shown in FIG. 7, the processing system 72 may in some embodiments further be coupled to one or more I/O devices such as a keyboard and/or mouse 76 and a display 74.

The generation software 78 includes a measurement data collection, registration, processing, and display module 80 that receives clinical and physiological information from a variety of measurement modalities described further below. The generation software 78 further comprises data selection and synthesis algorithms 86 that uses the measurements data gathered and processed by the module 80 to generate an output treatment map or lens contour specification 48d. In the embodiment of FIG. 7, which data is used and how that data is used by the data selection and synthesis algorithms 86 may be controlled and mediated through a user interface 82. In addition to allowing user selection of measurement modality data to use for the treatment map or lens contour specification 48d, the user interface may also control the use of thresholds 84 and a knowledge base 88 to allow user-friendly and intelligent treatment map or lens contour specification 48d generation that allows a wide variety of physician control and automated treatment map or lens contour specification 48d generation.

The measurement data collection, registration, and processing module 80 can receive measurement data about a subject's eyes from one or more of a variety of sources. In FIG. 7, measurement inputs 91, 92, 93, 94, and 95 are illustrated as connecting to module 80. It will be appreciated that from a hardware perspective, the measurement inputs to module 80 will physically enter the system via the processing system 72 which will store them in a memory in the processing system 72 where they can be accessed by the treatment map generation software 78. The measurement data used by the module 80 may be in a variety of formats and may enter the system in a variety of ways. For some data in some embodiments, relevant data can be entered manually into the computer system 70. This would typically be applicable for clinical data such as manifest refraction measurements, patient information such as age, gender, other clinical information about the patient, follow up results, etc. This information is designated 95 in FIG. 7. Other information potentially received by the module 80 involves outputs of measurement devices. Some of this data could be entered manually as well but in many embodiments the processing system 72 would be directly connected with, connected over a network (including possibly the Internet or Intranet) or even incorporated into one or more of the measuring instruments 91, 92, 93, 94. Another possibility is that data generated by one or more of the measuring instruments could be stored on a flash drive or other memory technology and transferred from a measuring instrument to the processing system 72 manually.

Turning now to the measurement modalities themselves, the system of FIG. 7 may comprise a corneal topology measuring instrument 91 such as Placido or Scheimpflug imaging, and a wavefront aberrometer 92 such as Hartmann-Schack, and an epithelial thickness measuring instrument 93, such as optical coherence tomography (OCT). Although OCT devices have been commercialized and made available, its use has been limited because of the lack of understanding of how the epithelium affects corneal laser ablation procedures and the lack of recognized medical protocols in that regard. The Inventor has performed clinical studies to better understand the effects of epithelial compensation on the anterior cornea surface and has developed the novel medical protocol, the CREATE™ Protocol to, among other things, assimilate epithelium mapping and OCT devices into new systems and methods for corneal laser ablation.

These three instruments 91, 92, and 93 may or may not include eye tracking functionality such as a optical imaging camera configured to establish eye markers for tracking and alignment of the maps generated by each of the devices. When included, such eye tracking functionality can help ensure proper registration of the images and measurements made by the devices so the outputs can be overlaid and appropriately compared and processed.

Other data gathering instruments that may be a part of the system include an axial eye length measuring instrument 94 which may be dedicated measurement hardware such as an interferometer but which may also be accomplished with OCT or other technologies. As noted above with respect to inputs 95, phoropter measurements may also be gathered to obtain manifest defocus and manifest astigmatism values for entry into the system. Although manifest refraction data would not necessarily be required when a wavefront aberrometer 92 is provided, it can still be useful for comparison with the objective measurements from the other instruments and in some cases a surgeon may want to perform laser ablation based on correcting the manifest refractive error.

As noted above, data from these instruments is collected, registered, and processed by treatment map or lens contour specification generation software 78. The registration and selection enables the epithelial thickness measurement and corneal topology measurement to be used concurrently for mapping of the anterior corneal surface. Both of these data sets can, for example, be referenced to the corneal apex determined independently with these measurements themselves, or a CCD camera can be used to determine pupil location during the measurements and the data sets can each be registered to that feature. An embodiment of this invention includes any particular referenced feature or features that enable the epithelial and topography measurements to be synced for registration and selection so measurement information can be used concurrently in a determination of an ablation pattern.

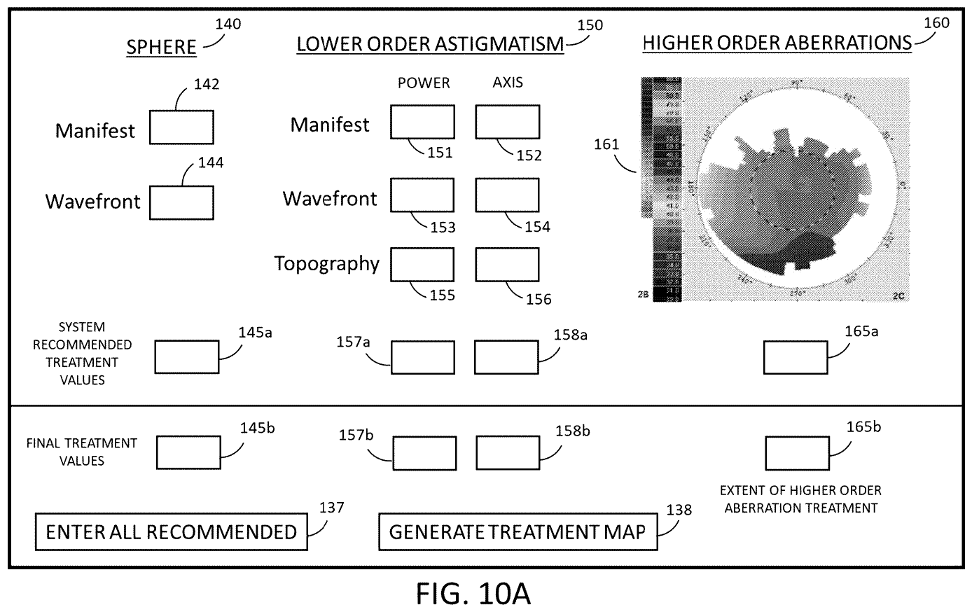

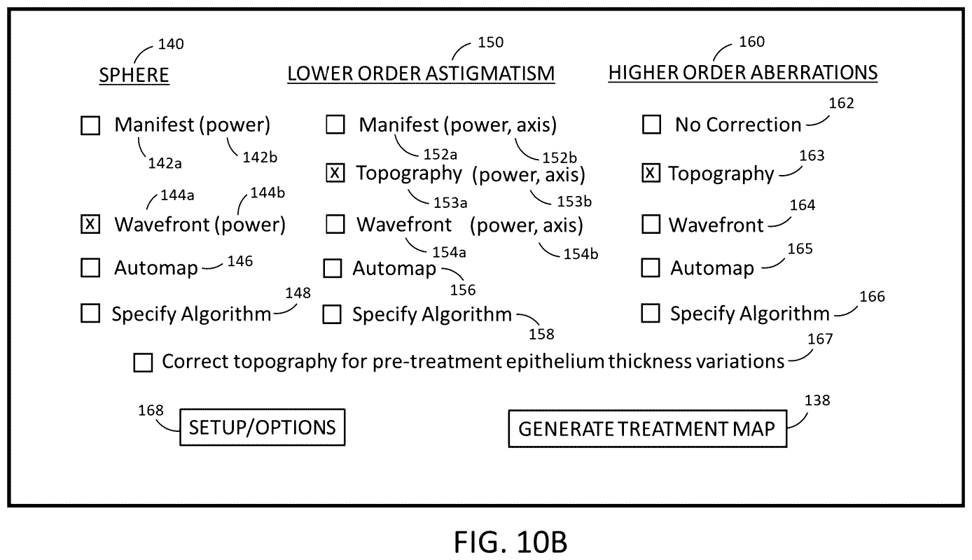

As described in more detail below, a system with the combination of data gathering technologies of FIG. 7 can provide a variety of treatment options for the optometrist, ophthalmologist, or surgeon. In conjunction with the data visualizations on the display 74, embodiments may include display of measurement modality results and a selection and/or entry of treatment options that can be provided on the user interface. One possible embodiment of such a user-interface is shown in FIG. 10A. Another is shown in FIG. 10B. The treatment map generation software may further comprise thresholds, other processing parameters, and a knowledge base 84 with content and use described further below. Using this information, and on automated analysis of the data from the measurement technologies, suggestions regarding the data on which to base the treatment map can be provided.

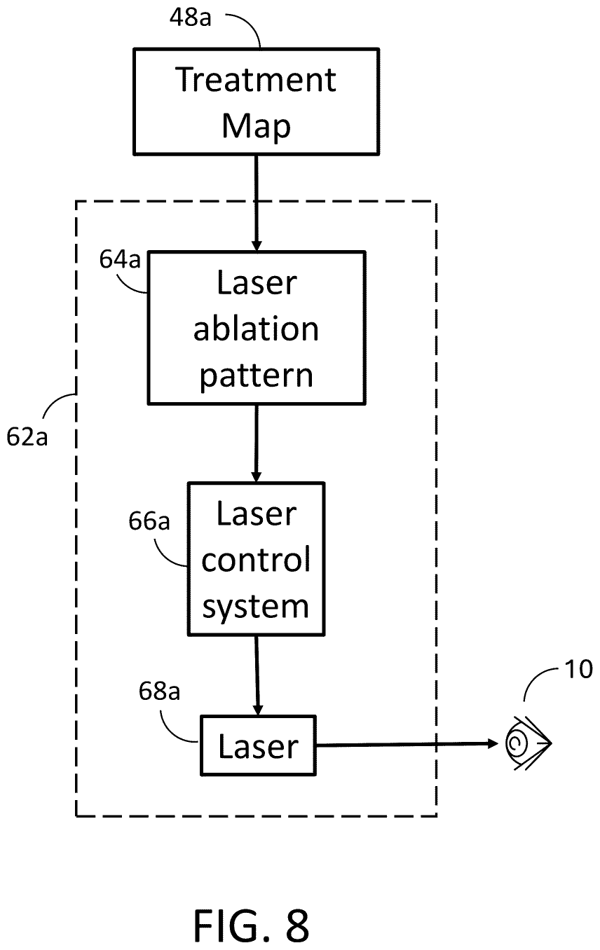

If the system of FIG. 7 is applied to laser ablation surgery such as PRK or LASIK, then as shown in FIG. 8, a treatment map 48d may be used as an input to a laser system 62 that performs the laser ablation accordingly. First, the treatment map 48d is used to generate a laser ablation pattern 64 which defines laser intensity, pulse duration, number of pulses, and other laser beam parameters necessary to accomplish the depths of corneal removal specified in the treatment map 48d. This laser ablation pattern 64 may also take into account such positional factors as the slope of the cornea at locations away from the apex. Because the laser ablation pattern 64 will need to take into account specific characteristics of the laser 68 being used in the system 62, software that generates the laser ablation pattern 64 from the treatment map 48d is typically closely associated with the laser system 62 itself. The laser ablation pattern 64 is used by a laser control system 66 to control laser position, pulse duration, and the like for the laser 68, which may, for example, be an excimer laser, to emit the appropriate laser pulses to perform the ablation surgery.

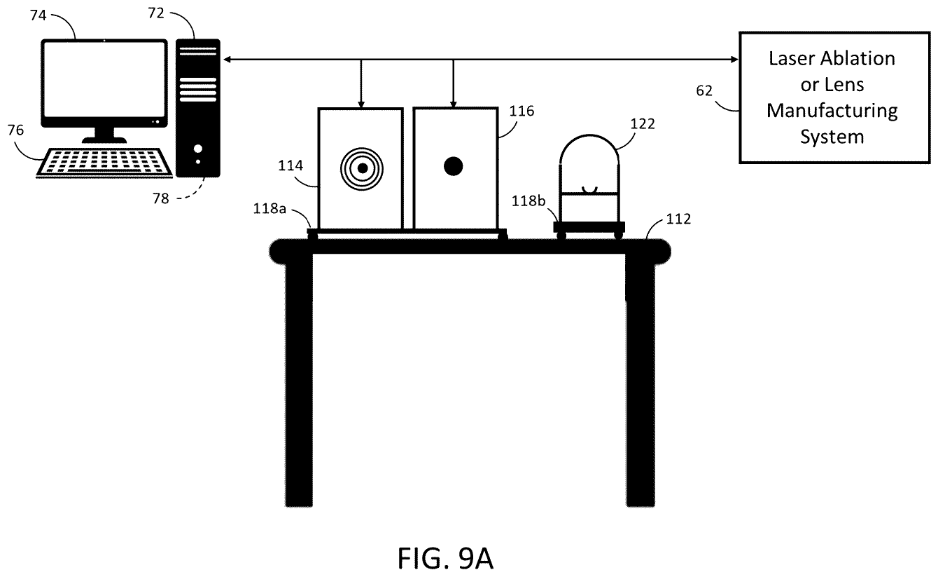

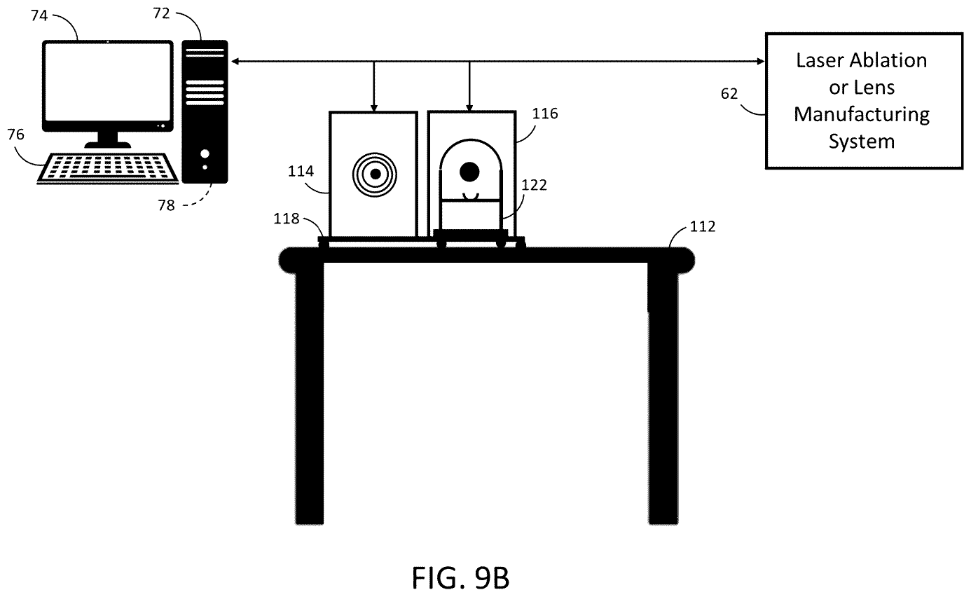

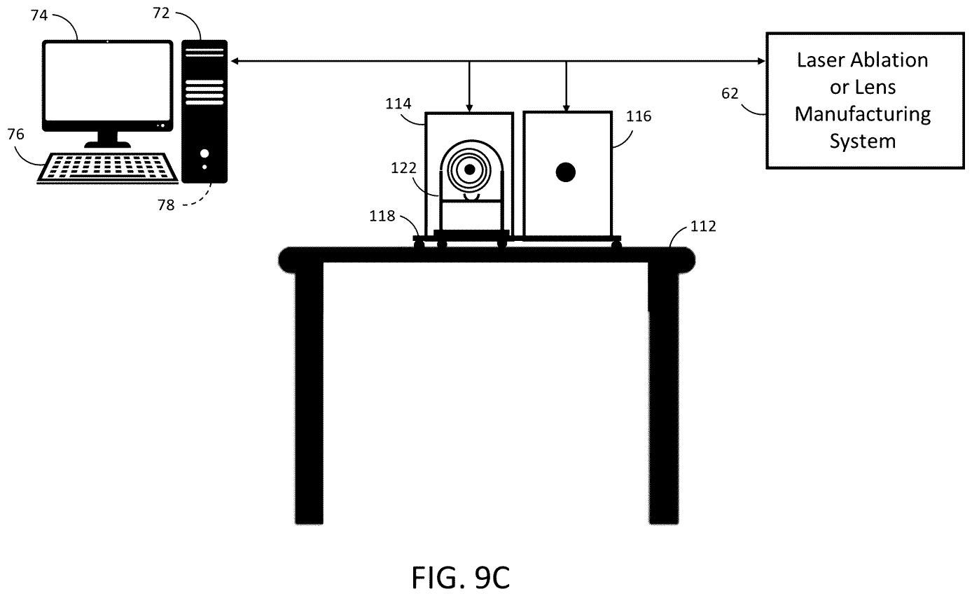

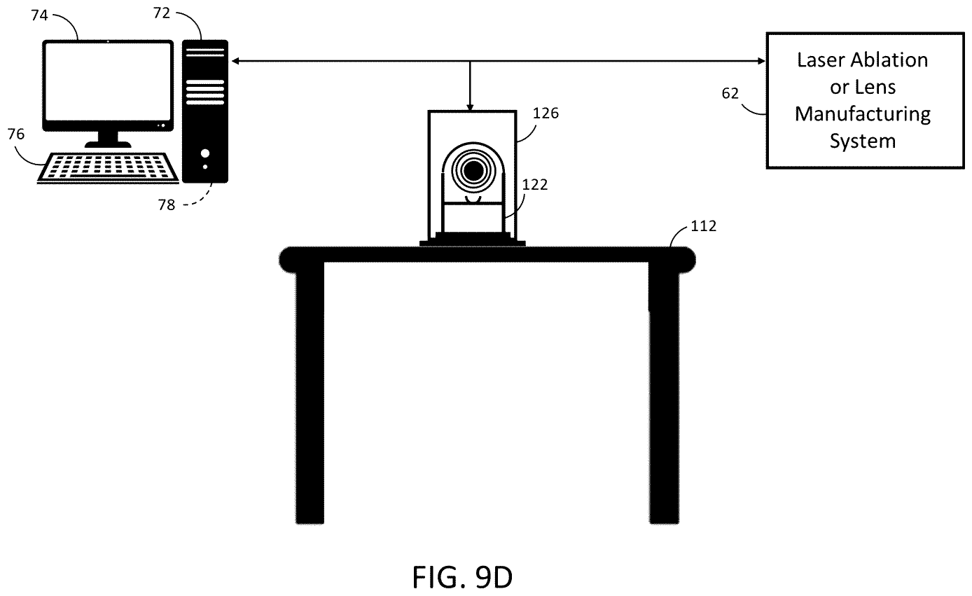

FIGS. 9A to 9C illustrate one exemplary embodiment of a corneal measurement and/or ablation system made in accordance with the principles described above with respect to FIG. 7 that may be in a surgeon's office or clinic for use with a patient. In this embodiment, a platform such as a table, bench, or cart 112 may support one or more measurement instruments 114 and 116 separately housed. A chin rest 122 to support the subject when placing their eyes in front of the instruments may also be provided. Any one or more of the instruments 114, 116 and chin rest 122 may have or be provided on wheels or wheeled carts 118a, 118b or may be otherwise moveable or slidable on the platform 112.

The instrument 114 may be a combined Placido ring corneal topography measuring device combined with a Hartman-Shack wavefront aberrometer. This combines instruments 91 and 92 of FIG. 7 into a single device. A commercially available example of such a device is the Schwind Peramis. Instrument 116 may be an OCT based epithelial mapping instrument such as the Optovue Solix. As shown in FIGS. 9B and 9C, the chin rest 122 and instruments 114 and 116 can be selectively moved in front of and away from the patient to make the measurements in a convenient manner with the subjects head being maintained substantially stationary during the process.

The instruments 114 and 116 are connected for data communication with a data processing system 132 with user display and I/O capabilities 51 such as described above. Also included is the laser ablation system itself 142. The processing algorithms for generating the ablation maps may be all in the processing system 132, all in laser ablation system 142, or distributed between them in any manner.

FIG. 9D illustrates a system similar to that shown in FIGS. 9A through 9C except in this embodiment a single instrument is a combined Placido ring topography measuring device, wavefront aberrometer, and OCT epithelium thickness measuring device.

FIG. 10A is an exemplary user interface that may be provided by software in some implementations of the system of FIG. 7. This user interface may be presented on the display 74 of FIG. 7. In this embodiment, three categories of aberration are presented in three different columns, where each column provides the ability to view different measurement modality results and determine the treatment or aspects thereof to be applied to each category of aberration.

The sphere column 140 may include an output at 142 which may show the outcome of a manifest measurement of the required myopia or hyperopia correction for the subject patient, performed, for example, with a phoropter as described above. This may be entered manually by the user directly into location 142 in this user interface or it may be received by the system through another user interface or another communication method. The sphere column 140 may also include an output at 144 that may show the outcome of a wavefront aberrometry measurement of the required myopia or hyperopia correction for the subject patient, performed, for example, with a Hartmann-Shack wavefront aberrometer as described above. This may be entered manually by the user directly into location 144 in this user interface or it may be received by the system through another user interface or another communication method, such as directly from the aberrometer via a communication channel. The sphere column may further include a location 145a that will show a system recommended correction for myopia or hyperopia that may be incorporated into the treatment map 48d. This value may, for example, be auto-filled by the system with the manifest sphere correction.

The lower order astigmatism column 150 may include outputs at 151 and 152 for lower order astigmatism power and axis correction values based on manifest measurements performed, for example, with a phoropter as described above. As with the manifest sphere correction this may be entered manually by the user directly into locations 151 and 152 in this user interface or it may be received by the system through another user interface or another communication method. The lower order astigmatism column 150 may also include outputs at 153 and 154 that may show the outcome of a wavefront aberrometry measurement of the required astigmatism power and axis correction for the subject patient, performed, for example, with a Hartmann-Shack wavefront aberrometer as described above. This may be entered manually by the user directly into locations 153 and 154 in this user interface or it may be received by the system through another user interface or another communication method, such as directly from the aberrometer via a communication channel. The lower order astigmatism column 150 may also include outputs at 155 and 156 that may show the outcome of a corneal topography measurement of the required astigmatism power and axis correction to form a spherical anterior corneal surface, performed, for example, with a Placido ring topographic measurement device as described above. This may also be entered manually by the user directly into locations 155 and 156 in this user interface or it may be received by the system through another user interface or another communication method, such as directly from the topography measurement device via a communication channel. The lower order astigmatism column 150 may further include locations 157a and 158 a that will show a system recommended correction for lower order astigmatism that may be incorporated into the treatment map 48d.

Determining an appropriate correction for lower order astigmatism to be used in the treatment map can be difficult, especially because the power and axis correction values determined by the different measurement modalities manifest, wavefront, and topography can be significantly different. Accordingly, advantageous embodiments of the system will auto-fill the locations 157a and 158a with a system recommended lower order astigmatism treatment. The Inventor's prior studies have shown that when the manifest astigmatism differs from the topography astigmatism value, better visual outcomes are usually obtained when the topography astigmatism value is used for the treatment map. A different issue arises however if the topography astigmatism value and the wavefront astigmatism value differ. The Inventor has created algorithms that may be used in such situation in order to determine whether POA exists and how it should be treated. If POA is determined via this algorithm to exist, then it may be advantageous to use all or part of the wavefront astigmatism measurement 153, 154 or a derivative of the topography and wavefront for generating the treatment map. The Inventor has created another algorithm to determine the measurement to be used to treat POA.

To auto-fill locations 157a and 158a with a system recommended lower order astigmatism treatment, the system may use a predefined rule for selecting between topography and wavefront or create a derivative of the topography and wavefront measurements for generating the astigmatism correction aspect of the treatment map. Such a rule may use the thresholds 84 shown in FIG. 7, comparing one or more differences between the topography astigmatism measurements 155 and 156 and the wavefront astigmatism measurements 153 and 154 to the thresholds to select between the topography measurements for astigmatism at 155 and 156 or the wavefront measurements for astigmatism at 153 and 154 to utilize when auto-filling locations 157a and 158a. The threshold for astigmatic power may, for example, be between plus or minus 0.1 and 0.75 diopters, and the threshold for astigmatic axis may, for example, be between plus or minus 1 and 15 degrees. The thresholds 84 may be predefined in the system, and the thresholds may be user settable through a part of the user interface 82. The rule used when the system auto-fills locations 157a and 158a may be to compare the topography power and axis 155, 156 with the wavefront power and axis 153, 154 and utilize the wavefront power and axis 153, 154 for the 157a and 158a entries if the difference for one or both the power and axis is greater than the respective threshold, and otherwise, use the topography measured power and axis 155 and 156 for the 157a and 158a entries. An alternative rule may, for example, be to use a weighted average of the topography correction 155, 156 and the wavefront correction 153, 154 for the entries at locations 157a and 158a. The weights may be equal, resulting in an astigmatism correction that is the average of the topographic correction 155, 156 and wavefront correction 153, 154. In other embodiments, the weights may be a function of the differences between the topographic astigmatism measurements 155, 156 and the wavefront astigmatism measurements 153, 154.

The system may additionally or alternatively use a machine learning algorithm to perform the auto-fill function for locations 157a and 158a. The system may use the knowledge base 84 of FIG. 7 to generate and/or update the rule used to perform the auto-fill function. In some embodiments, the system can employ a machine learning algorithm that takes as inputs one or more of the topographic, wavefront, manifest, OCT, and interferometer measurements as well as possibly other information about the patient such as age, gender, medical conditions, and the like. The machine learning algorithm could be trained to generate a recommended astigmatism treatment for auto-filling locations 157a and 158a based on these inputs using results obtained from prior patients in addition to the wavefront and topography measurements. Such a machine learning algorithm could be updated in real time by the knowledge base 84 as additional patients are treated and results for those procedures are added to the knowledge base.

The HOA are treated in this example system based on the corneal topography data. Corneal topography data is advantageous because it is high resolution and use of this data for treating higher order aberrations generally leads to smoother more spherical corneal surfaces following surgery which as described above the inventor has found to be especially advantageous. The HOA column 160 may provide a display of a corneal elevation map 161. The HOA column 160 may also provide a location 165a with a system recommended value for how many orders of correction depth the treatment map should be designed to treat. This entry 165a may be auto-filled with a default value such as 3, which may specify a treatment map with corrections for trefoil and coma higher order aberrations. Although corneal topography is believed at this time to be the superior choice for treating HOA with this system, alternative systems using wavefront data for HOA are also possible.

The user interface may also include a set of corrections that will be incorporated into the treatment map 48d. A selection button 137 may be provided that populates the sphere correction 145b, lower order astigmatism correction 157b, 158b, and higher order correction depth 165b with the system recommended values. If desired by the surgeon, one or more of these system recommended values may be overridden by the user manually entering different values. For example, based on the user's review of the data in locations 142 and 144 the user may enter an average or other derivative of the information provided in the locations 142 and 144 into location 145b. Similarly, the surgeon may be allowed to override the recommended lower order astigmatism treatment recommended by the system by, for example, clicking on locations 157b and 158b and entering different values as determined by the judgment of the surgeon. If desired, the user may enter a different value such as 4 or 5 in location 165b to correct additional or fewer higher order aberrations. After the values for the treatment map at locations 145b, 157b, 158b, and 165b are populated to the satisfaction of the surgeon, a generate treatment map button 138 may be selected to generate the treatment map in accordance with the populated values 145b, 157b, 158b, and 165b.

FIG. 10B is an exemplary menu style user interface that may be used in some implementations of the system of FIG. 7. In this implementation, the user is given nearly complete control of how the different measurement modalities contribute to the final treatment map. As with the user interface of FIG. 10A, this user interface may be presented on the display 74 of FIG. 7. It will be appreciated that a given system for corneal laser ablation may provide only a subset of the user options provided in FIG. 10B. As shown in FIG. 10B, a collection of check box choices 130 for laser ablation treatment map 48d creation may be presented to the physician or other user. In this embodiment, as was the case with the embodiment of FIG. 10A, three categories of aberration are presented in three different columns, where each column provides the ability to select the treatment to be applied to each category of aberration.

The sphere column 140 allows as two of the options a correction for myopia or hyperopia with a manifest measurement 142a or a wavefront measurement 144a. To assist in making a decision, the sphere correction power that will be applied from manifest measurement 142b and the sphere correction power 144b that will be applied from wavefront measurement can be presented to the user on the same display. Another option for sphere correction may be provided by an “automap” option 146. This option may cause the system to select the sphere correction for the treatment map in an automated matter. The system selection may correspond to a predefined rule for selecting between manifest and wavefront or deriving a correction from the manifest and wavefront measurements. The automap function may use the knowledge base 84 of FIG. 7 to generate and/or update the rule used to perform the automap 146 sphere correction determination. In some embodiments, the automap function can employ a machine learning algorithm that takes as inputs one or more of the topographic, wavefront, manifest, OCT, and interferometer measurements as well as possibly other information about the patient such as age, gender, medical conditions, and the like. The machine learning algorithm could be trained to generate a treatment map based on these inputs using results obtained from prior patients. Such a machine learning algorithm could be updated in real time by the knowledge base 84 as additional patients are treated and results for those procedures are added to the knowledge base. More flexibility may be provided by a “specify algorithm” option 148. If this box is selected, the user may be taken to a different screen which allows the user to select from a set of sphere correction algorithm options to choose from. A predefined selectable algorithm may, for example, be the average of the manifest and wavefront corrections. The system may also allow the user to create alternative algorithms for their own use that once defined by the user and entered into the system may be selected for sphere correction via the “specify algorithm” checkbox 146.

The lower order astigmatism column 150 may allow as three of the options a correction for astigmatism with a manifest measurement 152a, a topography measurement 153a, or a wavefront measurement 154a. To assist in making a decision, the astigmatism correction power and axis that will be applied from manifest measurement 142b, topography measurement 153b, and wavefront measurement 154b can be presented to the user on the same display. In general, these three astigmatism values will differ from each other. The Inventor's prior studies have shown that when the manifest astigmatism differs from the topography measured astigmatism value, better visual outcomes are usually obtained when the topography measured astigmatism value is used for the treatment map. A different issue arises however if the topography measured astigmatism value and the wavefront measured astigmatism value differ significantly. This situation often indicates that astigmatism is arising from posterior sources such as the lens. In this situation, it may be advantageous to use the wavefront astigmatism measurement 154a, 154b or some other variant of the wavefront measured astigmatism measurement for generating the treatment map. With the display shown in FIG. 10B, the power and axis values 153b provided for the topography measurement and the power and axis values 154b provided for the wavefront measurement can be compared by the user. If the user concludes from such a comparison that the difference between the topography power and/or axis 153b is different from the wavefront power and/or axis 154b by an amount that is large enough that posterior astigmatism is indicated, the user may select the wavefront measurement 154a for use in generating the treatment map or use a variant of the two measurements. The user may have certain threshold differences in power and/or axis in mind when making the decision about whether to use topography 153a, wavefront 154a, or a variant for the astigmatism correction aspect of the treatment map.

As with the sphere correction, another option for lower order astigmatism correction may be provided by an “automap” option 156. This option may cause the system to select the astigmatism correction for the treatment map. The system selection may correspond to a predefined rule for selecting between topography and wavefront or creating a derivative of the topography and wavefront measurements for generating the astigmatism correction aspect of the treatment map. The automap function may use the thresholds 84 shown in FIG. 7, comparing one or more differences between the topography astigmatism measurement 153a and the wavefront astigmatism measurement 154a to the thresholds to select between the topography measurement for astigmatism 153a or the wavefront measurement for astigmatism 154a when generating the treatment map. The threshold for astigmatic power may, for example, be between plus or minus 0.1 and 0.75 diopters, and the threshold for astigmatic axis may, for example, be between plus or minus 1 and 15 degrees. The thresholds 84 may be predefined in the system, the thresholds may be user settable through a part of the user interface 82. The algorithm used when the automap function 156 is selected for astigmatism may be to compare the topography power and axis 153b with the wavefront power and axis 154b and utilize the wavefront power and axis 154b for the treatment map if the difference for one or both the power and axis is greater than the respective threshold, and otherwise, use the topography measured power and axis 153b. The automap function may also use a machine learning algorithm such as described above with the sphere correction. Also as with the sphere correction, more flexibility may be provided by a “specify algorithm” option 158 for the astigmatism correction. If this box is selected, the user may be taken to a different screen which allows the user to select from a set of astigmatism correction algorithm options to choose from. A predefined selectable algorithm may, for example, be a weighted average of the topography correction 153b and the wavefront correction 154b. The weights may be equal, resulting in an astigmatism correction that is the average of the topographic correction 153b and wavefront correction 154b. In other embodiments, the weights may be a function of the differences between the topographic astigmatism measurement and the wavefront astigmatism measurement. The system may also allow the user to create alternative algorithms for their own use that once defined by the user may be selected for astigmatism correction via the “specify algorithm” checkbox 146.

A column 160 for selecting correction strategies for HOA is also provided in the example user interface of FIG. 10B. A first option 162 of no correction for HOA is provided in this embodiment. This may be selected if the surgeon wishes to treat a subject using only traditional manifest data. In such an instance, a surgeon may select manifest 142a for sphere, manifest 152a for lower order astigmatism, and no correction 162 for higher order corrections. Similar to the sphere column 140 and lower order astigmatism column 150, the HOA option column 160 provides the option to select higher order corrections based on topography measurements at 163 or wavefront measurements 164. Also similar to the sphere correction column 140 and the lower order astigmatism correction column 150 another option for HOA correction may be provided by an “automap” option 165. This option may cause the system to select the HOA correction for the treatment map in an automated matter. The system selection may correspond to a predefined rule for selecting between topography and wavefront or deriving a correction from the topography and wavefront measurements. The automap function may use the knowledge base 84 of FIG. 7 to generate and/or update the rule used to perform the automap 165 HOA correction determination. The automap function 165 may also use a machine learning algorithm such as described above with the sphere correction. More flexibility may be provided by a “specify algorithm” option 166. If this box is selected, the user may be taken to a different screen which allows the user to select from a set of HOA correction algorithm options to choose from. A predefined selectable algorithm may, for example, allow the surgeon to correct only certain Zernicke aberrations such as only coma, but not correct other HOA. Other options for specialized algorithms available from this option 166 may be to correct some HOA based on wavefront measurements and other HOA based on topography measurements. The system may also allow the user to create alternative algorithms for their own use that once defined by the user may be selected for the HOA correction via the “specify algorithm” checkbox 166.

An option can also be provided in the menu interface of FIG. 10B for epithelial compensation in the treatment for differences in pre-treatment epithelium thickness at selection 167. This selection can incorporate information from the epithelium thickness measuring instrument 93 of FIG. 7. This epithelial compensation can be incorporated into the treatment map in different ways depending on whether a PRK or LASIK procedure is being performed. If PRK is being utilized, laser ablation of the epithelium to a depth of approximately the thickest part of the epithelium in the treatment zone may be performed. This will cause ablation of stromal tissue as well in those areas where the epithelium is thinner than the thickest portion. This will alter the corneal topography because some raised portions will be lowered and these changes can be taken into account in topographically guided corrections for HOA, astigmatism, and defocus that are incorporated into the ablation map. If a LASIK procedure is being performed, a local point by point subtraction of epithelium thickness from the topographically measured height values can be performed; which results in an accurate topographic map of the stromal tissue itself. After lifting the flap, the ablation map can provide ablation in accordance with the epithelium corrected topology. After flap replacement, the stromal surface will be smoother, resulting in a more even epithelium layer after healing. This option 167 can be selected or not in association with any combination of selections from the correction columns 140, 150, or 160 described above.

In some embodiments, the automap and/or specify algorithm options of FIG. 10 may include the option of using ray tracing algorithms for generating the treatment map. Ray tracing has been experimented with for over a decade and it continues to have several significant flaws in the methodology as in its existing use. Problems with a ray tracing methodology that uses wavefront include the following: (1) It continues to follow the scientific postulation that posterior ocular astigmatism is present in a significant number of patients; therefore, requiring “whole” eye measurements and the use of data responsible for suboptimal outcomes. It ignores research by the Inventor that demonstrates that the majority of suboptimal outcomes have nothing to do with posterior ocular astigmatism, but rather other factors, such as, epithelial compensation and LASIK flap induced corneal shape change, (2) It ignores the fact that crystalline lens changes could affect the outcome of the measurements, as the lens changes as a person ages. This could make data in patients over the age of 50 inaccurate and create an incorrect laser profile, (3) It uses conventional and existing understanding of the epithelial layer, which is not considered to be variable in the normal Gullstrand models of the eye; in other words, it uses data that considers the epithelium to be an even thickness blanket over the anterior cornea following the shape of the cornea underneath. As such, existing methodologies do not add or change a wavefront-based map for changes in epithelial thickness. The existing raytracing systems have in documented studies shown to continue to increase HOA.

Embodiments as described herein that provide epithelium thickness information can be used to improve ray-tracing algorithms by taking this thickness information into account when developing the ray tracing eye model for treatment map generation.

Another interface feature illustrated in FIG. 10B is a setup/options button 168. Selecting this button may cause display of one or more user interface screens through which the surgeon can, for example, set the thresholds 84 in FIG. 7 that may be used in the automap function 156 and define algorithms that are available for treatment map generation using the select algorithm options 148, 158, and 166.

After the surgeon makes their selections from columns 140, 150, and 160, they can select the button 169 to initiate the generation of the treatment map in accordance with the selections.

The menu driven treatment map generation interface of FIG. 10B allows a surgeon to generate treatment maps using a wide variety of clinical protocols. As mentioned above, for example, a traditional pure manifest treatment map can be generated by selecting options 142, 152, and 162. A wavefront guided treatment map can be generated by selecting options 144, 154a, and 164. A topographic guided treatment map can be generated by selecting options 142, 153a, and 163. As will be described further below with some specific examples, the system can allow optimal use of the different measurement modalities, especially the topography and wavefront measurement modalities, which provide both overlapping and complementary information regarding the optical characteristics of a subject eye. Depending on the measurements themselves, their quality, and/or their availability, either topographic information alone, wavefront information alone, or a selective combination of topographic and wavefront information can be used based on the surgeon's judgement for each particular patient.

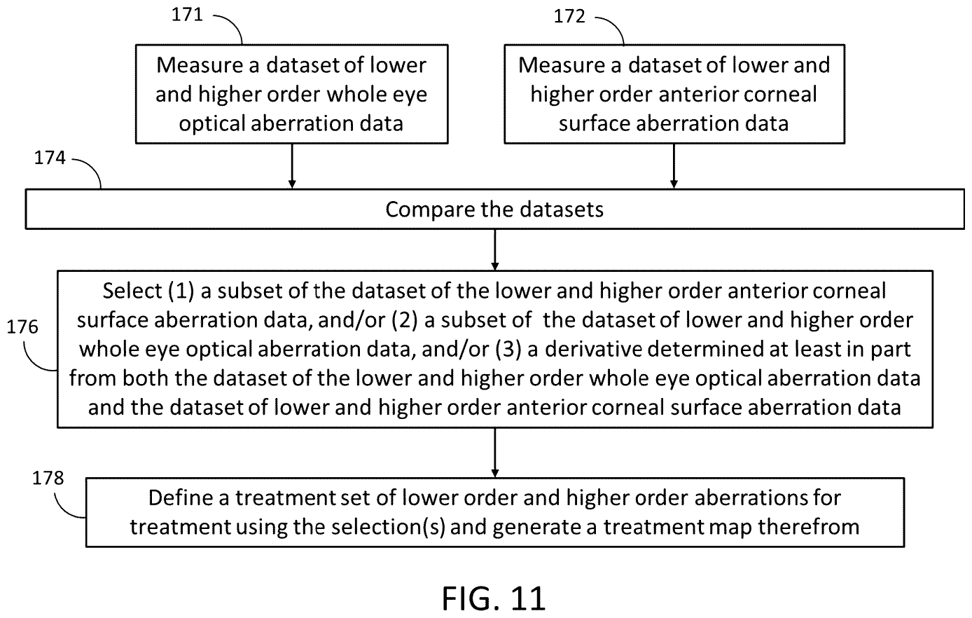

FIG. 11 is a flowchart that illustrates a method of selection of one or both of topographic information and wavefront information by a surgeon or other user of the system that may be enabled and facilitated by the systems of FIGS. 7-10B described above. Referring now to this FIG. 11, at block 171 a dataset of lower order and higher order whole eye optical aberration data is measured. At block 172 a dataset of lower order and higher order anterior corneal surface aberration data is measured. At block 174, the two datasets are compared. Based at least in part on the comparison performed at block 174, a selection from multiple options is performed. As shown in block 176, a subset (which may be any portion of or all of the dataset of block 171) of the whole eye optical aberration data may be selected, and/or a subset (which may be any portion of or all of the dataset of block 172) of the anterior corneal surface aberration data may be selected, and/or a derivative determined at least in part from both the whole eye optical aberration data and anterior corneal surface aberration data may be selected. At block 178, based at least in part on the selection(s) made, a treatment set of lower order and higher order aberrations for correction are defined, and a treatment map is generated therefrom.

In some cases, more than one of the options of block 176 can be used for generating a single treatment map. In addition, other factors such as manifest measurements and/or epithelium thickness related measurements can be used as part of generating a treatment map along with the selections of the options in block 176 that are illustrated in FIG. 11. For example, a surgeon may, after comparing the datasets at block 174, decide to follow the first option of block 176 and use topographic measurements for lower order astigmatism and higher order aberrations and use manifest measurements for sphere. In this case, the second and third options of block 176 are not used, and the wavefront measurements of block 171 may be ignored for the generation of the treatment map. For another patient, a surgeon may select both the first and second options of block 176, using, for example, wavefront measurements for sphere and topographic measurements for lower order astigmatism and higher order aberrations. Another example of using both the first and second options of block 176 for generating a single treatment map is using the wavefront measurements for both sphere and lower order astigmatism and using topographic measurements for higher order aberrations.

The third option of block 176 comprises selecting a derivative determined at least in part from both the dataset of lower and higher order whole eye optical aberration data and the dataset of lower and higher order anterior corneal surface aberration data. Such a derivative can take a variety of forms and be generated in a variety of ways. Different algorithms for computing such derivatives can be available to the surgeon through the specify algorithm options 148, 158, and 166 of FIG. 10 for example. In one example derivative computation, the lower order astigmatism component of the whole eye optical aberration data and the lower order astigmatism component of the anterior corneal surface aberration data can be combined in selected percentages as a weighted average. If the weights are selected equal, then 50% of the correction indicated by the whole eye lower order astigmatism is added to 50% of the correction indicated by the anterior corneal surface aberration data. As another alternative to this scenario, the weights could be adjusted depending on the difference between the whole eye astigmatism aberration correction and the anterior corneal surface aberration astigmatism correction. For example, a large difference between whole eye astigmatism and corneal surface astigmatism may indicate that astigmatism is arising from posterior sources such as the lens. To take this into account, an algorithm may increase the weight of the whole eye aberration astigmatism relative to the anterior corneal surface astigmatism as the difference between the whole eye astigmatism and the anterior corneal surface astigmatism increases. As described above, another alternative algorithm may weight the anterior corneal surface aberration astigmatism at 100% and the whole eye astigmatism aberration at 0% unless the difference between the whole eye aberration astigmatism and the corneal surface aberration astigmatism is greater than a threshold, in which case the anterior corneal surface aberration astigmatism is weighted at 0% and the whole eye aberration astigmatism is weighted at 100%. This type of algorithm can be conceptualized as using the anterior corneal surface aberration data astigmatism as a default starting point, and then modifying or replacing that astigmatism value with the whole eye aberration astigmatism data under appropriate conditions that may be pre-defined or evaluated on-the-fly by the surgeon. A derivative may also be generated from a machine learning algorithm such as described above. All of the computations, selections, derivatives, and the like described herein applied to lower order astigmatism could also be applied to higher order aberrations such as coma and trefoil.

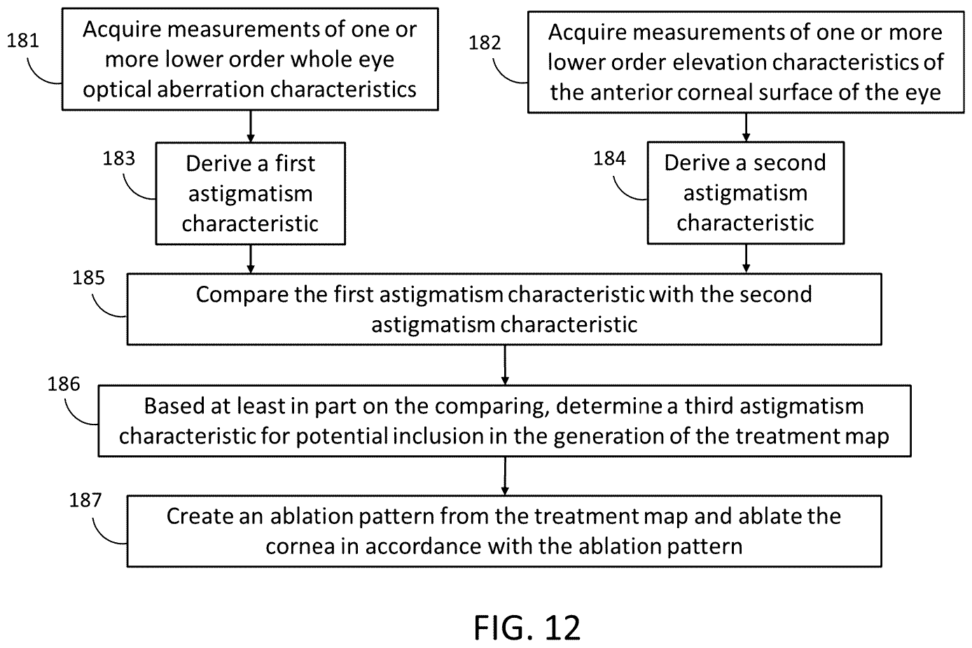

FIG. 12 is an illustration of one method of treating astigmatism according to some embodiments. The method of FIG. 12 is another example of a treatment protocol or portion thereof that may be performed in accordance with the several options set forth in block 176 of FIG. 11 and is especially applicable to the embodiment shown in FIG. 10A. Referring now to FIG. 12, at block 181 measurements of one or more lower order whole eye optical aberration characteristics are acquired, and at block 183 a first astigmatism characteristic is derived therefrom. This first astigmatism characteristic may correspond to the entries in locations 153 and 154 of FIG. 10A. At block 182, measurements of one or more lower order anterior corneal surface elevation characteristics are acquired, and at block 184 a second astigmatism characteristic is derived therefrom. This second astigmatism characteristic may correspond to the entries in locations 155 and 156 of FIG. 10A. At block 185, the first and second astigmatism characteristics are compared. At block 186, based at least in part on the comparing, a third astigmatism characteristic is determined for potential inclusion in the generation of a treatment map. This third astigmatism characteristic may correspond to the entries in locations 157a and 158a and/or the entries in locations 157b and 158b of FIG. 10A. At block 187, an ablation pattern is created from the treatment map, and the subject cornea is ablated in accordance with the ablation pattern. As described above, the third astigmatism characteristic can be generated from the first and second astigmatism characteristics in a variety of ways.

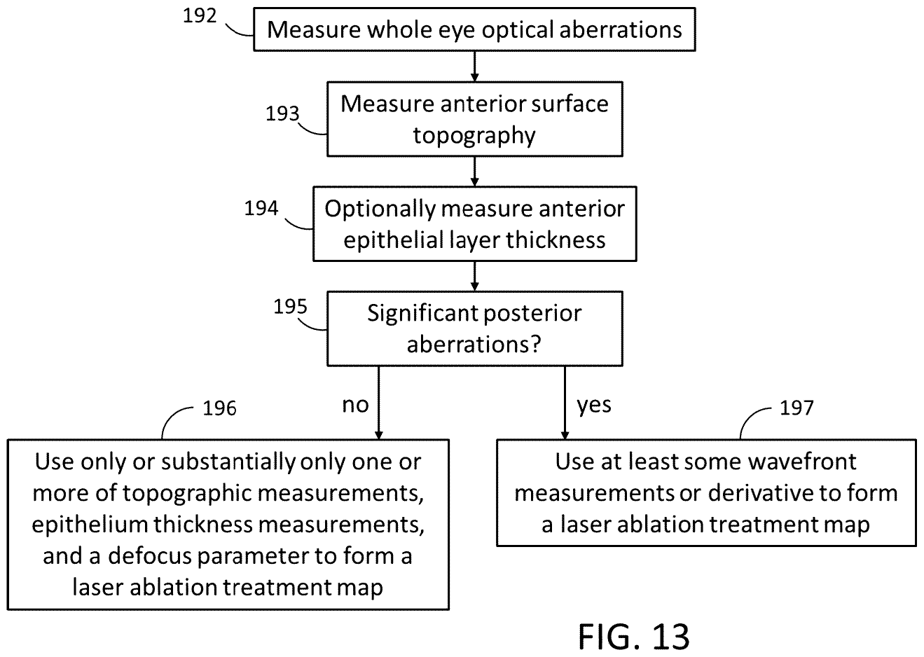

Referring now to FIG. 13, another exemplary method is illustrated. The method of FIG. 13 is another example of a treatment protocol or portion thereof that may be performed in accordance with the several options set forth in block 176 of FIG. 11. In this method, at block 192, whole eye optical aberrations are measured. This may be performed with the wavefront aberrometer 92 of FIG. 7. At block 193, anterior surface topography of the eye is measured, such as with the topology measuring instrument 91 of FIG. 7. At block 194, epithelium thickness is optionally measured, such as with the epithelium thickness measuring instrument 93 of FIG. 7.

At block 195, a determination is made regarding the distribution of the aberrations of the eye between characteristics of the anterior ocular portion and the posterior ocular portion. This determination may be more specifically directed to the question of whether characteristics of the posterior ocular region are a significant cause of the measured optical aberrations for the whole eye which can be measured with, for example, the wavefront aberrometer. By comparing the whole eye aberration data gathered at block 192 with the anterior surface topography data of block 193, information regarding the separate contribution of anterior and posterior ocular structures to the detected refractive aberrations for the whole eye can be determined. For example, both the wavefront aberrometer data and topography data may be processed to generate independent astigmatism correction values to be performed with the laser ablation. As shown in FIG. 13, if these values are similar, it can be concluded at block 195 that posterior structures do not contribute significantly to the astigmatic aberrations of the eye. In this case, it can be advantageous to follow the “no” path of FIG. 13 to block 196 and to use only or substantially only one or more of topographic measurements, epithelium thickness measurements, and a defocus parameter to form a laser ablation treatment map. This pathway performs laser ablation in a manner that will form a smooth, close to spherical contour for the anterior corneal surface, without attempting to create an imperfect anterior corneal surface that compensates for theoretical posterior sources of optical aberration. If, however, the whole eye astigmatism measured with the wavefront aberrometer differs from the astigmatism present on the anterior surface of the eye as measured topographically then a determination must be made if there are astigmatic aspects to posterior ocular structures such as the posterior corneal surface and/or the lens. In some cases when they differ there may be no or very little POA and in other cases there may be significant POA. The Inventor has developed an alogorithm to detect when POA is present in those cases. If POA is present, the method can follow the “yes” path to block 197, where at least some wavefront aberrometer measurements or derivative are used to form a treatment map or lens contour specification 48.

Following the “yes” path may involve using wavefront aberration measurements for any one or more than one aberration correction. In some situations, only wavefront astigmatism may be used as part of the treatment map and the rest of the corrections could be based on topography and manifest measurements. In some situations, data from the wavefront aberrometer 92 may be the primary or even sole source of data used to generate a treatment map or lens contour specification 48.

### Glasses and Contact Lenses

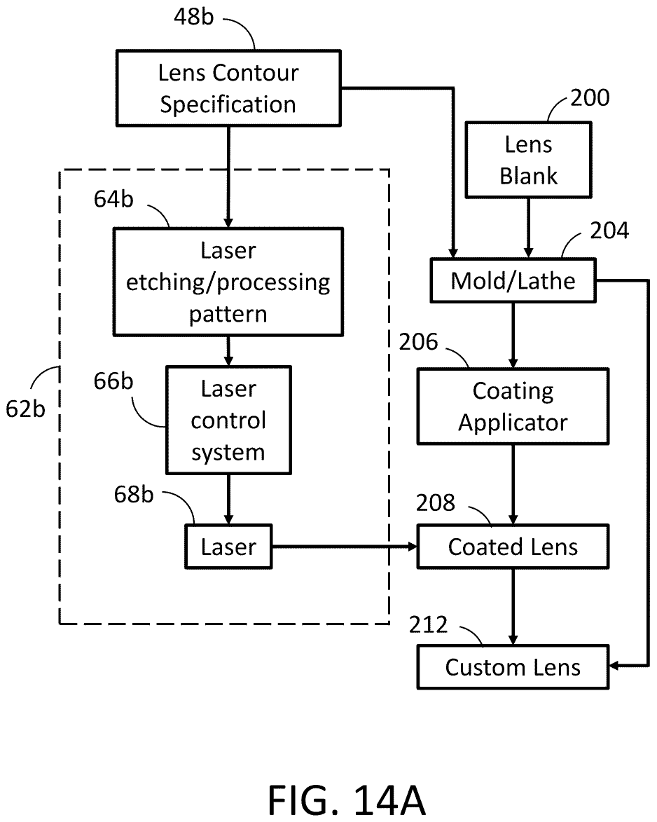

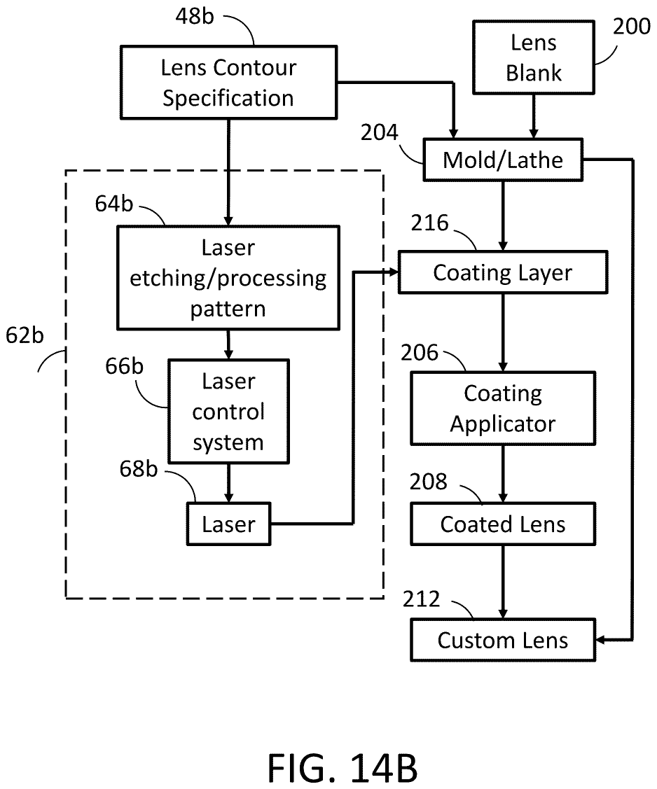

FIGS. 14A and 14B show another use for the systems and methods described herein. The systems of FIGS. 14A and 14B are analogous to that shown in FIG. 8, except instead of a corneal laser ablation treatment map 48a, a lens contour specification 48b is generated, which may be used as an input to a laser system 62b portion and/or a mold/lathe 204 portion of a lens manufacturing process. A laser etching/processing pattern 64b derived from the lens contour specification 48b may be used by a laser control system 66b to control laser position, pulse duration, and the like for the laser 68b to emit the appropriate laser pulses in accordance with the lens contour specification 48b. In the systems of FIGS. 14A and 14B, instead of performing ablation on a cornea, laser etching/processing is performed to customize a lens for glasses or contact lenses to correct HOA of the wearer's eyes which may be determined by wavefront aberrometry or by corneal topography. In some embodiments, the selection of which to use may be performed using the methods and systems described above.

As shown in FIG. 14A, a lens blank 200 may be processed by a mold/lathe 204 to generate the desired corrections for lower order aberrations myopia, hyperopia, and lower order astigmatism. These corrections may be derived from the lens contour specification 48b. A lens blank 200 may not be provided depending on the manufacturing method used, especially for contact lenses which may be molded directly at the outset with the desired lower order corrections. Following molding or lathing, the lens is then processed by a coating applicator 206 which affixes a coating layer to the lens, which may be on the back side facing the cornea in use. The coating layer may be thin, for example less than two millimeters thick, advantageously less than one millimeter thick, and may be designed thin enough and with an appropriate index of refraction to not significantly affect the optical performance of the lens as it exists prior to application of the coating layer. The coating layer of the coated lens 208 may then be laser processed with the laser 68b in accordance with the lens contour specification 48b to imprint corrections for the subject's HOA into the coating layer. The laser may etch the layer material with the higher order aberration corrections. Another alternative is to use a layer material that is heat modifiable, where the imprinting of the HOA correction is done by selective heating and local deformation of the coating layer. The output of the process is a custom lens 212 which incorporates corrections for both lower order and higher order aberrations. In some embodiments, the molding/lathing performed at block 204 can implement both the lower order and higher order corrections defined by the lens contour specification, and the laser processing of a coating layer is not performed. Although not shown in FIG. 14A or 14B, it is also possible that the coating layer is processed mechanically, chemically, thermally, or by another means to incorporate the HOA corrections instead of by a laser as shown.

As shown in FIG. 14B, another option is to etch/process a coating layer as described above with HOA corrections before it is attached to a lens that has been processed to provide the sphere and astigmatism corrections. Thus, with the systems of FIGS. 14A and 14B, glasses and contact lenses can be manufactured from the lens contour specifications generated with the above-described systems and methods which include custom corrections for HOA.

### Intraocular Lenses and the Implantation Thereof

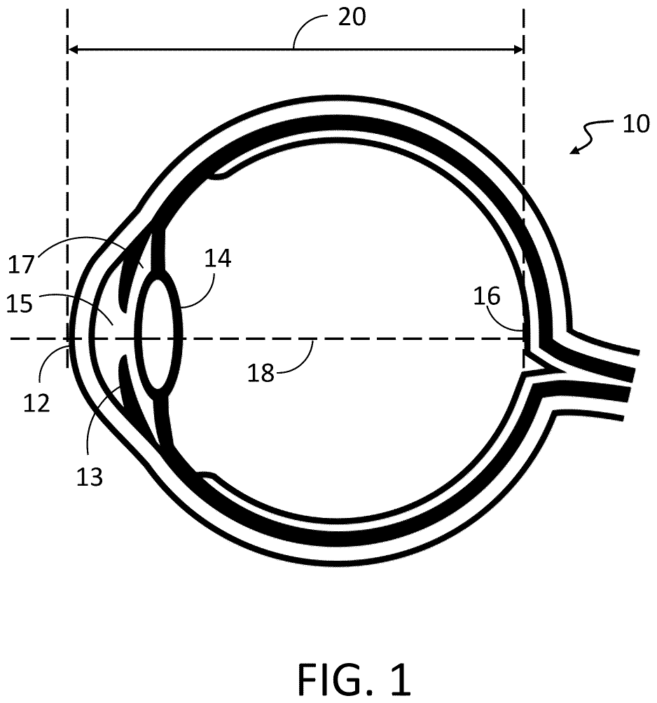

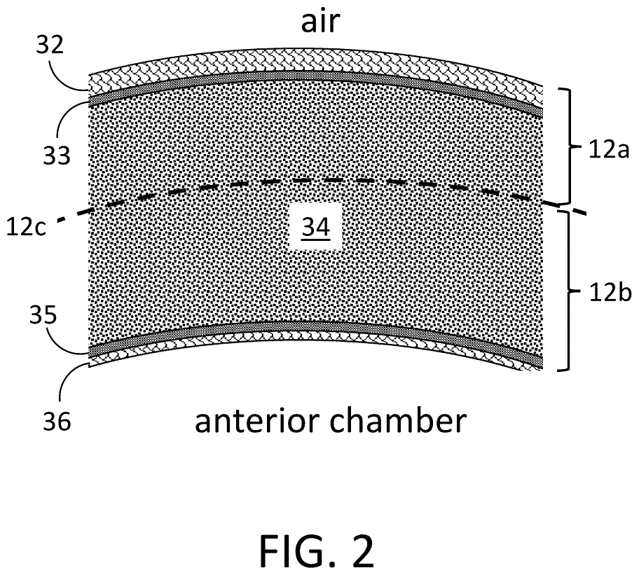

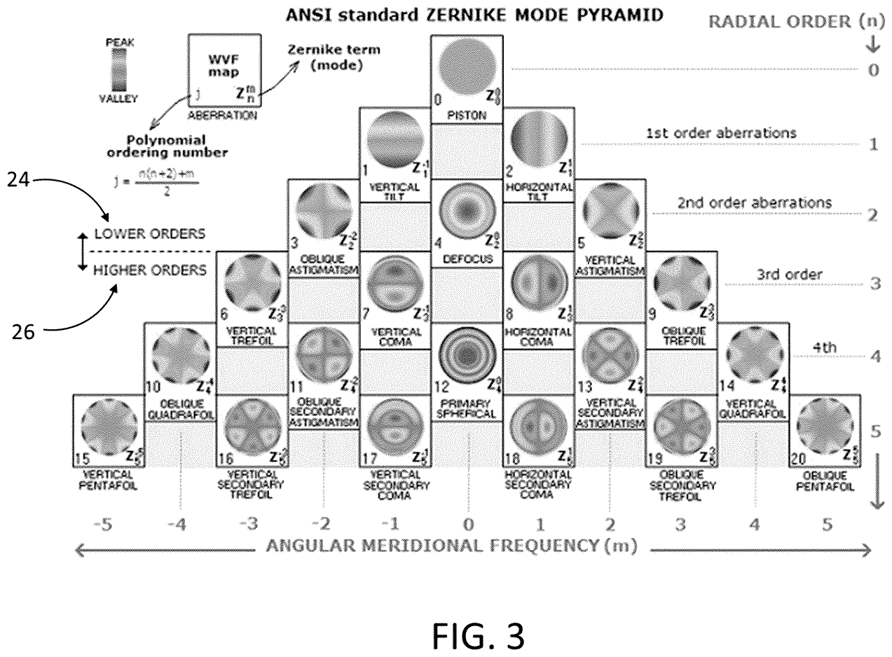

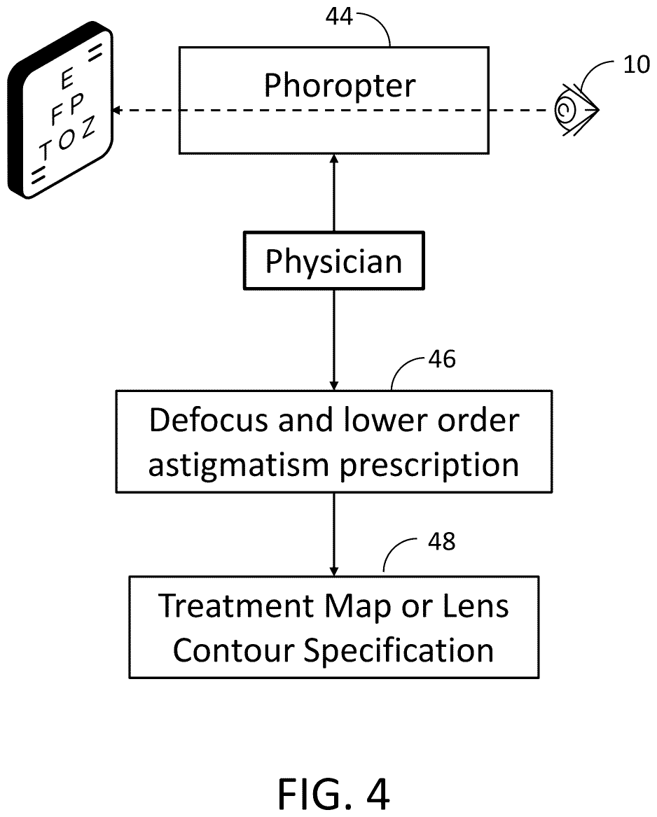

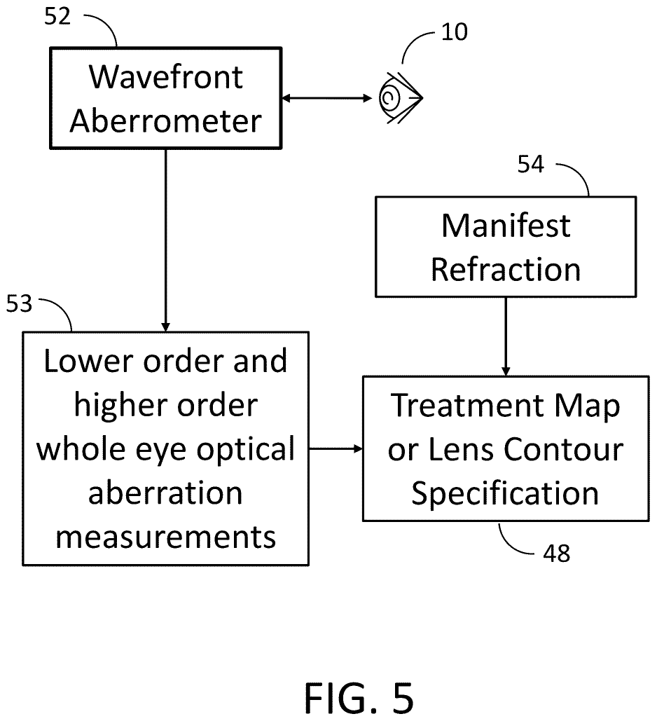

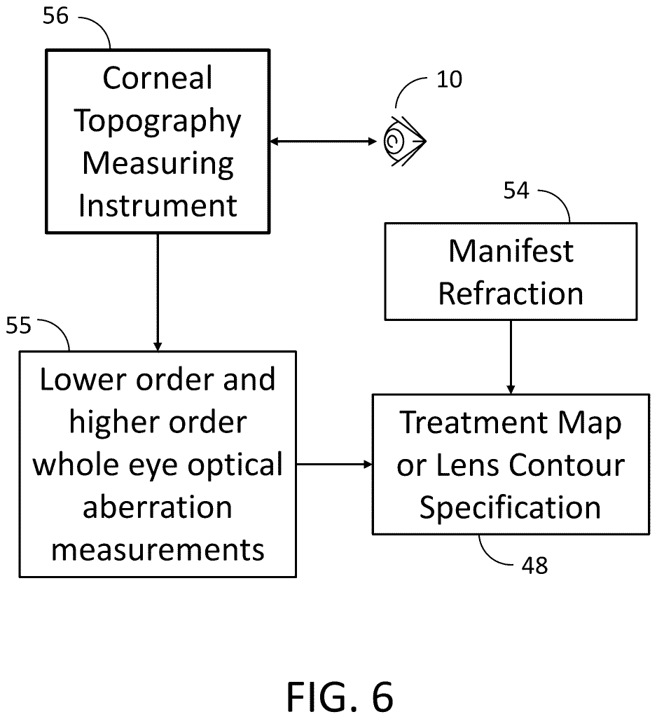

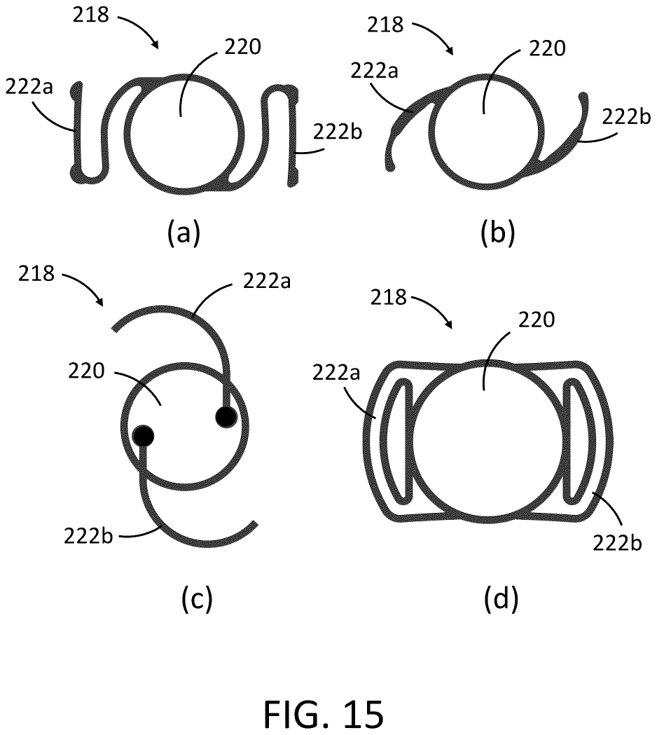

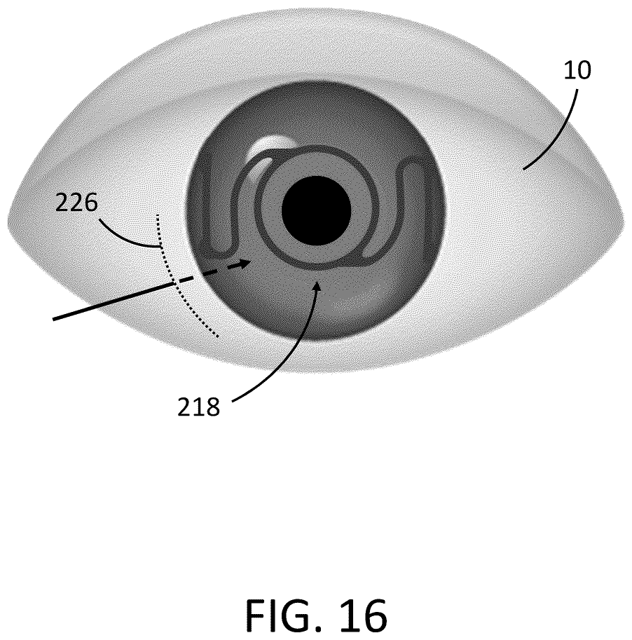

Intraocular lenses (IOLs) are implanted under the cornea either in the anterior chamber 15 (FIG. 1) between the cornea and the iris or in the posterior chamber 17 (FIG. 1) between the iris and the lens 14 (FIG. 1). In these cases, the natural lens itself may or may not be removed. The intraocular lens can also be placed inside the capsular sac of a lens that is removed during the implantation procedure or is otherwise not present in the eye of the subject. FIG. 15 shows four IOL 218 designs designated (a), (b), (c), and (d). Intraocular lenses generally comprise a central lens (optic) portion 220 and opposing haptic portions 222a and 222b that are configured to mechanically hold the intraocular lens in position after implantation. Referring now to FIG. 16, an IOL 218 is inserted through an incision 226 somewhat to the side of the pupil and iris 10, over the sclera for example. As mentioned above and discussed further below, IOLs can be implanted over an existing natural lens or can be used as a replacement for a lens that is absent or removed during the implantation procedure. In FIG. 16, the IOL is shown positioned in the anterior chamber, anterior to the iris.

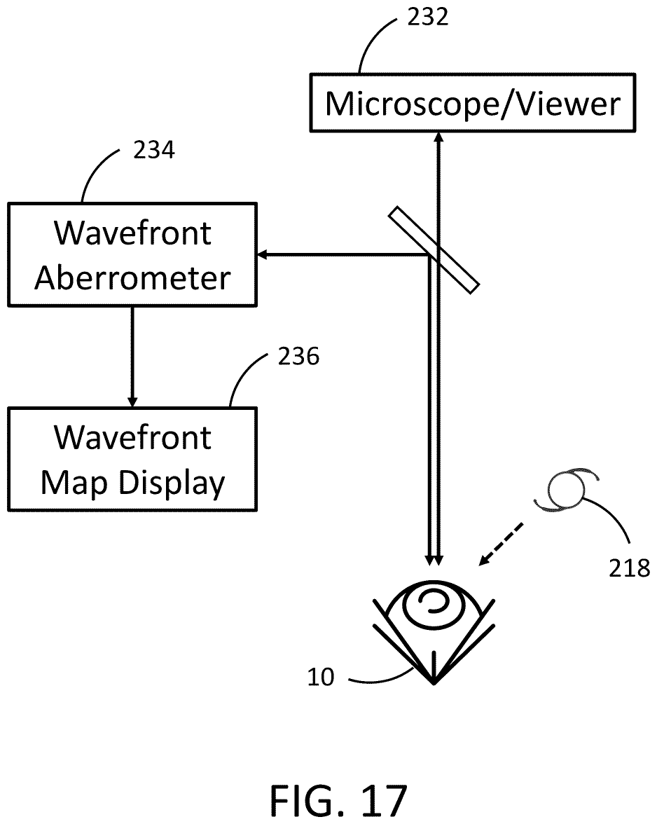

Intraocular lenses can be used to correct lower order sphere and astigmatism aberrations and have also been created with extended depth of focus for presbyopia treatment. When IOLs include astigmatism correction, proper rotational orientation of the astigmatism correction axis is required for optimal performance. However, this can be surgically difficult to perform precisely under a surgical microscope, and misalignments can lead to the need for a secondary repositioning procedure or the use of glasses as an additional correction modality after implantation of the IOLs. FIG. 17 illustrates one technique for assisting proper placement of an IOL that includes corrections requiring precise placement. In the system of FIG. 17, structured light from a wavefront aberrometer 234 (e.g. Hartmann-Shack) is injected into the optical path of the surgical operating microscope 232. As in conventional wavefront aberrometry, the reflection from the retina is collected. Another display 236 is provided that shows the map of residual aberrations after the IOL is installed. During the procedure, the position and/or orientation of the IOL in the eye can be adjusted while monitoring the wavefront map on the display. The IOL can be adjusted intraoperatively to minimize the residual aberrations on the display 236.

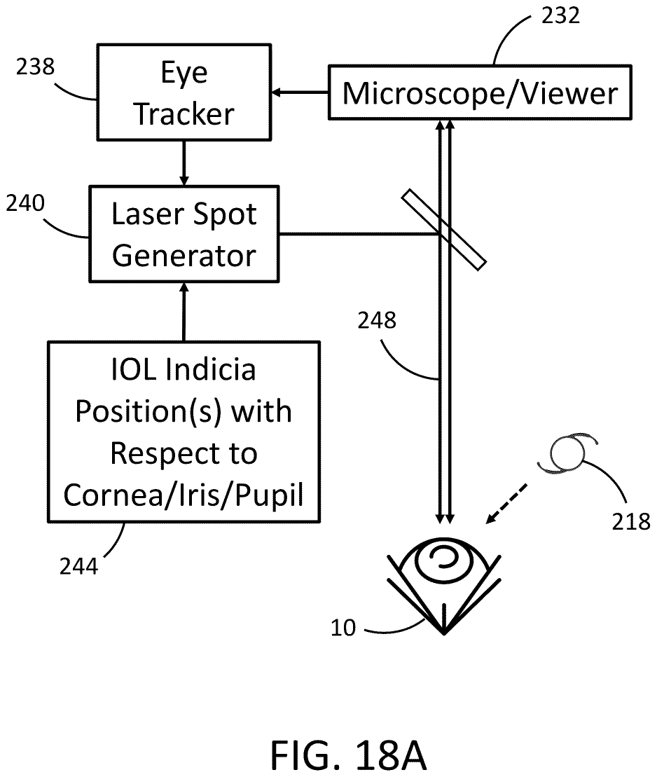

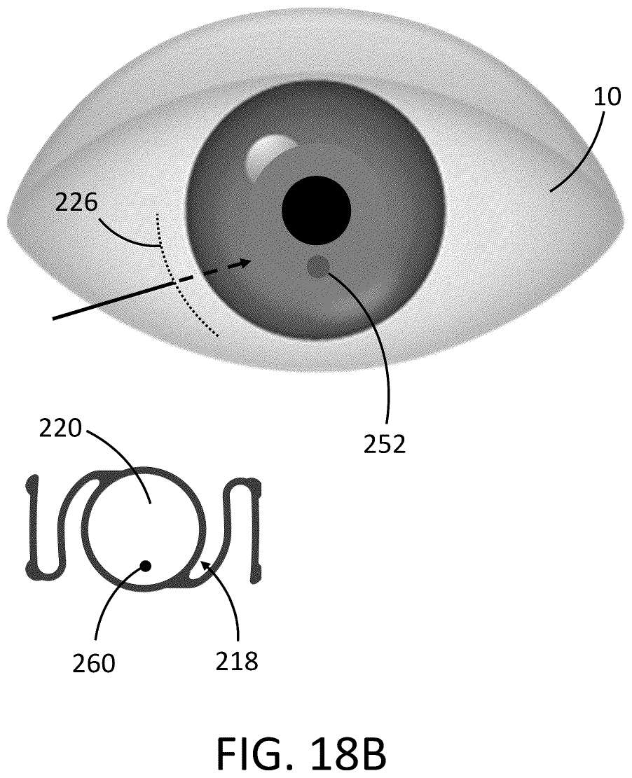

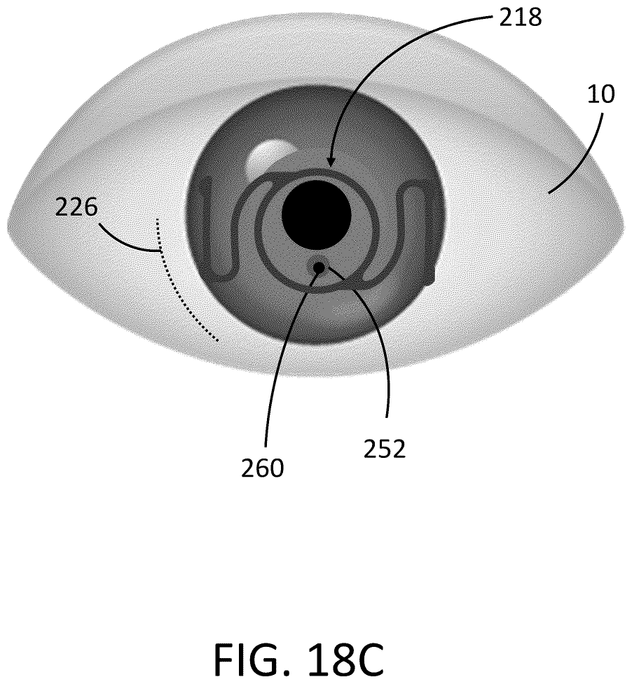

However, intraoperative adjustment with this method can be time consuming and difficult to accurately achieve intraoperatively. FIGS. 18A through 18C illustrate an alternative positioning system and methodology that helps address these issues. Referring now to FIGS. 18A through 18C, a laser beam 248, such as from a low power helium-neon laser beam generated by a laser spot generator 240 is injected into the optical path of the microscope/viewer 232. This beam 248 makes a laser spot 252 (FIG. 18B) on the cornea surface at a location which is visible in the field of view of the microscope/viewer 232. In addition, the intraocular lens 218 includes an indicia 260, etched, marked, or otherwise affixed to a portion thereof. This indicia bears a known relationship to the aberration corrections formed in the lens portion 220 of the IOL such as the axis of any provided astigmatism correction. As shown in FIG. 18C, during the surgery, the IOL is placed such that the indicia 260 is aligned with the visible laser spot 252 in order to ensure accurate placement and orientation of the IOL 218. Referring back to FIG. 18A, as illustrated at blocks 238 and 244 the laser spot generator takes information defining the desired position of the IOL indicia 260 with respect to the eye (e.g. with reference to the iris and/or pupil) when the IOL is properly positioned and oriented along with data from a conventional eye tracker 238 to continually position the laser spot 252 at the location where the IOL indicia 260 should be for the IOL 218 to be properly positioned and oriented. By aligning the indicia 260 with the laser spot 252 intraoperatively, proper placement of the IOL is relatively easily accomplished.

It is one advantage of this system that it can be implemented in an automated, semi-automated, or robotic manner if the microscope/viewer acquires digital image data of the surgical area. If the microscope/viewer comprises, for example, a CCD camera, image processing and analysis can identify and locate the laser spot 252 and indicia 260 in the acquired digital images of the surgical area. A robotic arm with an end effector grasping, for example, a haptic of the lens could be programmed to push or otherwise manipulate the lens to align the indicia 260 with the laser spot 252. Such a robot may or may not also perform the surgical incision into which the lens is inserted.

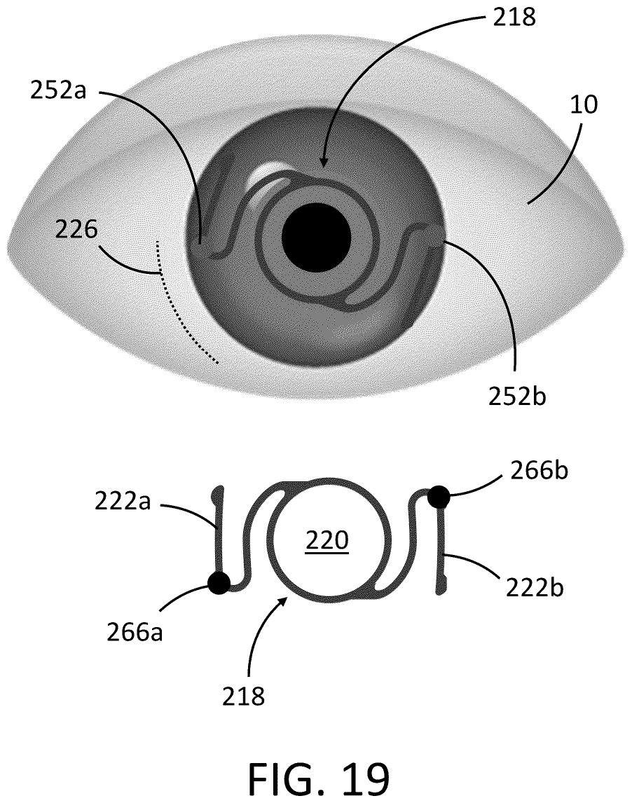

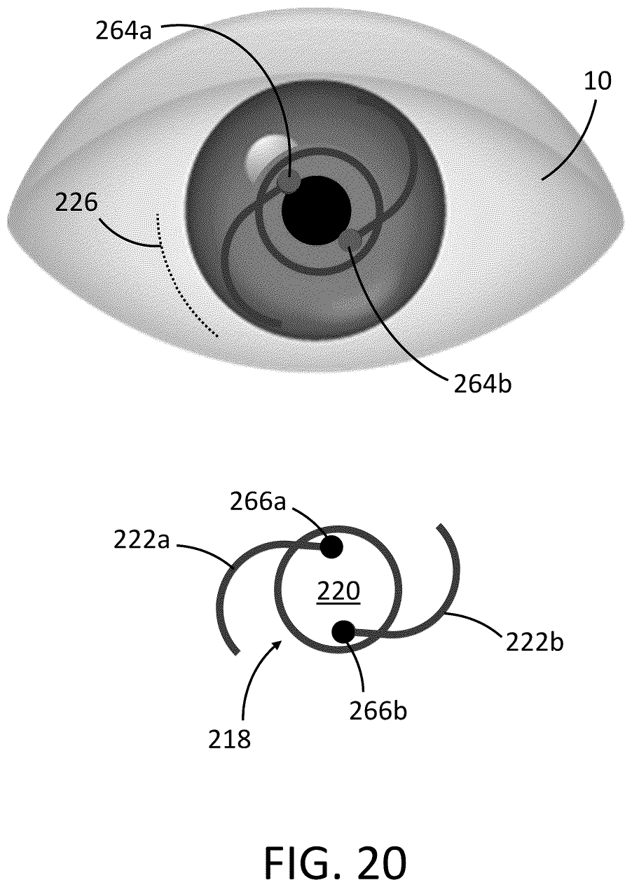

As illustrated in FIG. 19, it is possible to use two laser spots 264a and 264b to be aligned with two indicia 266a associated with the IOL 218. In this implementation, the two indicia 266a, 266b could be associated with the lens portion 220 of the IOL or as shown in FIG. 19 they could be associated with the haptic portion 222a, 222b. FIG. 20 shows a similar configuration but with a multi-part IOL where the laser spots 252a and 252b are to be aligned with the points at which each haptic wire 222a, 222b attaches to the lens portion 220 of the IOL. In this embodiment, no separate dedicated indicia markings or etchings would be required. Instead, the indicia 266a and 266b are inherent and visible portions of the IOL construction. This is possible as well with one-piece IOLs with integral haptics where corners, notches, bumps, or other constructional features of the haptics portion 222 and/or lens portion 220 could be defined as the feature or features of the IOL to align with the one or more laser spots 252.

The IOL embodiments of FIGS. 18 through 20 are implanted in the anterior chamber which is convenient because the markings on the IOL will be visible through the cornea and over the top of the iris. The systems and methods described here can also be used on IOLs implanted in the posterior chamber or lens capsular sac. In these cases, the lens portion 220 of the IOL could be marked closer to its central region and viewed through the pupil. In these cases, the surgery could be performed under pupil dilation so that the indicia are normally blocked by the iris after surgery. Another alternative is to use indicia that fade or dissolve over time to become invisible after the surgery. The above-described placement technique is especially useful if corrections for higher order aberrations are incorporated into the lens (optic) portion of the IOL. In this case, accurate vertical placement, lateral placement, and rotational orientation are all important to optimize aberration correction and thus lens performance and post-surgical visual acuity. All three of these positioning parameters can be accurately realized with the techniques described above, thereby enabling the incorporation of HOA correction into intraocular lenses which has not been previously accomplished.

### Cataract Treatment With Intraocular Lenses

Treatment of cataracts involves removing a clouded natural lens of the eye and replacing it with an intraocular lens such as those described above. Determining appropriate corrections for an IOL intended to replace a clouded natural lens is especially difficult because the cloudiness of the lens interferes with many measurement modalities. The commonly used technique for mapping the eye, wavefront measurement, has the advantage of measuring whole eye lower order aberrations (including whole eye astigmatism) but must also measure whole eye HOA. In cataract patients the clouding of the lens and vitreous changes corrupt the overall HOA data and make wavefront unacceptable to use in someone needing cataract surgery. In contrast, the above-described techniques for astigmatism and HOA correction are based upon the usual use of topography data which is not corrupted by cataract formation, and only uses the wavefront data or a derivation to determine the treatment of lower order astigmatism when POA is present and this portion of the wavefront data is not corrupted by lens changes or vitreous changes. Furthermore, the above-described IOL positioning techniques provide accurate vertical, lateral, and rotational alignment with the cornea to provide a more homogenous image on the retina, allow more light to pass, prevent any color shift due to prismatic effect of the aberrations which would lead to crisper, clearer, and brighter vision. As noted above, an extended depth of focus can be provided in the IOL to alleviate presbyopia which functionality is further improved by canceling corneal HOAs as less light would be scattered decreasing the impact of the increased spherical aberration created for the increased depth of focus.

It may be noted that the systems and methods described herein can be used to determine a specific value for lens astigmatism. Posterior ocular lower order astigmatism and higher order aberrations (those posterior to the anterior corneal surface) has four main components when the light path is followed. These are the posterior corneal surface which can be measured by Scheimpflug imaging which has been shown to cause small amounts of astigmatism, the lens which cannot be imaged or measured directly but is likely responsible for those patients with significant posterior ocular astigmatism, the vitreous, which may add aberrations but not astigmatism through syneresis but this cannot be measured, and the retina, which may cause small amount of astigmatism which is measurable by OCT imaging. If posterior ocular astigmatism is defined as any astigmatism posterior to the anterior cornea, this leaves posterior corneal astigmatism, lenticular astigmatism, and retinal astigmatism as sources. With the multi-imaging format systems and methods described above, if a patient has wavefront measured astigmatism which is different than the topography measured astigmatism, then the difference is likely due at least in part to posterior ocular astigmatism. If posterior corneal astigmatism is then measured with Scheimpflug, and retinal astigmatism with OCT, a value for lenticular astigmatism can be calculated. This may be very useful for cataract surgeries. Cataract surgeries involve replacing a patient's lens with a replacement lens implant. If the lens being removed has a known amount of astigmatism, reproducing the patient's pre-surgical vision quality after surgery would require the implant to have a similar astigmatism as part of its design. In some cases of cataract surgery, the new lens implant is designed to correct the pre-surgical eye myopia, hyperopia, and astigmatism. When this is being done, knowing how much astigmatism is being removed by taking out the natural lens can be used to better design the implant to correct the remaining sources of astigmatism.

### Intraocular Lens Manufacturing

The current ecosystem of IOLs, while advanced, is fundamentally limited in its ability to address patient-specific HOAs. Standard IOLs are primarily designed to correct lower-order aberrations, such as myopia and astigmatism, with toric IOLs specifically addressing corneal astigmatism. While some premium IOLs are designed to reduce spherical aberration, they still rely on a standardized, mass-produced design that cannot account for the unique dips and valleys on an individual patient's cornea and lens. A mass-produced lens, even with a key focal point, could be misaligned with an HOA on a patient's eye, potentially making the vision issues feel even more noticeable.

Attempts at a more customized approach are underway but represent an incomplete solution. The RxSight Light Adjustable Lens (LAL) is a notable innovation that allows surgeons to fine-tune the lens power and astigmatism post-surgery via a UV light treatment. This acknowledges the critical need to adjust for post-operative lens shift and refractive changes, but it does not address the full spectrum of HOAs. Similarly, the Atia Vision OmniVu lens, a shape-changing IOL currently in clinical trials, aims to restore the full range of functional vision but operates on a fixed-power front optic. These solutions represent important steps but fall short of a truly personalized HOA-correcting system. The market is waiting for a solution that can account for both the static irregularities of the eye's shape and the dynamic shifts that occur during healing. Our methods and systems can assist in providing this solution, which starts with our CHOICE™ (Customized HOA Optical Integrated Created Enhancement) IOL production system.

The novel eye enhancement methods and systems described herein move the field of ophthalmology towards profound transformation. In particular, these new methods and systems can move ophthalmology beyond mass-produced, standardized IOLs toward a future of true personalization, providing superior vision and quality of life for people. The novel manufacturing processes can provide end-to-end systems for the manufacturing and delivery of novel HOA-correcting IOLs that are capable of correcting all known types of higher-order aberrations.

Making the manufacturing process for these novel HOA-correcting IOLs economically viable requires a shift in current production paradigms. Current IOLs are mass-produced to keep costs down. These novel HOA-correcting IOLs would be custom made, which requires either an entirely new manufacturing process or an effective modification to existing manufacturing processes. The inventor has designed multiple types of new systems for manufacturing our novel HOA-correcting IOLs. Our preferred system integrates the key features of the systems described herein. The preferred system may be referred to as CHOICE™, which is a custom HOA optical integrated created enhancement system that includes the key features for manufacturing novel HOA-correcting IOLs. The first system is an enhancement on today's processes (i.e. mass produced IOLs), that either adds a coating to the pre-made lenses that has the corrective HOA pattern embedded in the coating or capable of being etched into the coating or etches the HOA pattern via laser into the IOL lens itself. The second system is an even more novel approach whereby a system combines advanced imaging, a centralized design platform, and just-in-time, laser-based manufacturing. This approach shifts the cost from a high-volume, inventory-based model to a highly precise, on-demand, and personalized production process. The third system is a combination of the above two systems that incorporate the efficiencies of today's processes (i.e. mass produced IOLs) with the centralized design platform, and just-in-time, laser-based manufacturing. The fourth system is additive manufacturing (i.e. 3D printing). By using biocompatible materials and high-precision 3D printers, the system could create IOLs with the exact surface topography needed to correct all HOAs. This would similarly shift the cost from a high-volume, inventory-based model to a highly precise, on-demand, and personalized production process. Some embodiments of the systems include one or all of those described below.

Advanced Patient-Specific Imaging and Data Capture: The process begins with a comprehensive scan of the patient's eye to determine lower and higher order aberrations. This process of determining lower and higher order aberrations and a comprehensive measurement could include the other novel methods and systems described herein, including our NuClarityVision™ system. This data would become the “blueprint” for the production of our novel HOA-correcting IOL. Integrated System: The captured data could then be sent to a secure, integrated central system. The system could have a cloud-based platform and/or central computing system.

Specified IOL design: The system could then be programmed to provide a custom specific HOA-correcting pattern based on the particular measurements. Artificial Intelligence design: The system could alternatively be programmed to use Al to create a “digital twin” of the patient's eye. The Al then could simulate the ideal IOL design, accounting for the unique HOA signature, corneal topography, and other anatomical factors. Such system could account for predictable post-healing shifts and changes.

Just-in-time production model: The system could integrate a “just-in-time” production model, where a patient's prescription is communicated directly to the manufacturer for immediate fabrication, or put into a queue for fabrication.

Laser-Based Subtractive Manufacturing: Instead of traditional lathe cutting and molding, the custom IOLs could be manufactured using a high-precision, laser-based subtractive process, potentially one of the core novelties of a system. A standardized, pre-cured IOL blank (or a pre-cured specialized IOL with pre-determined astigmatic corrections) would be placed in a manufacturing cell, and the laser would then alter the material with nanometer precision to create the custom-made optical surfaces required to correct the patient's specific HOAs. This process is highly repeatable and eliminates the need for polishing, further reducing production costs and time. The use of a standardized blank (or pre-cured pre-determined lower order astigmatism and/or spherical corrections) for all lenses allows for economies of scale on the raw material side.

In addition, a novel use of adaptive and diffractive optics can be used in manufacturing our specialized HOA-correcting lenses. While they don't replace our lens production systems and methods, they offer unique capabilities for correcting aberrations, reducing system size and weight, and creating complex light patterns.

Adaptive optics (AO) is a technology that uses deformable mirrors or other components to correct real-time distortions in an optical system's wavefront. While AO isn't typically used to manufacture the final lens itself, it's a critical tool for improving the performance and manufacturing processes of high-precision optical systems.

Key embodiments include (1) Correcting aberrations—In our novel manufacturing processes, AO systems can be used to compensate for optical aberrations caused by imperfections in a person's eye. This allows for the creation of sharper, more accurate images, which is essential for retinal imaging and designing custom corrective lenses (2) Improving laser material processing—In our novel manufacturing processes, AO can be used to correct for wavefront errors in high-power laser systems, which can be caused by heat or imperfections in the optical train. This ensures the laser beam remains perfectly focused and stable, leading to more precise and efficient cutting, welding, or other material processing tasks; and (3) Fabricating aspheric lenses—Aspheric lenses have a non-spherical surface to correct for aberrations, but they're difficult to manufacture with high precision. Adaptive optics can be integrated into the fabrication process to monitor and correct the shape of the lens surface in real-time, ensuring a more accurate and uniform final product.

Diffractive optics are components that manipulate light by using diffraction, the bending of light waves as they pass around an obstacle. These elements have a patterned surface, often with micro-or nano-scale structures, that a computer-generated design determines.

Key embodiments include: (1) Assisting the creation of thinner lenses—Unlike traditional refractive lenses that are thicker, diffractive lenses can be made thinner. This can significantly reduce the size and weight of a lens; (2) Correcting chromatic aberration—Chromatic aberration is a color distortion that occurs when different wavelengths of light are focused at different points. Diffractive optical elements (DOEs) have a unique property where their dispersion is the opposite of that of a refractive lens. By combining a diffractive element with a refractive lens, manufacturers can effectively cancel out chromatic aberration and create a more achromatic (color-corrected) system; (3) Beam shaping and splitting—DOEs can be designed to split a single laser beam into multiple beams or reshape the beam's intensity profile into a specific pattern. This can assist in providing specialized HOA patterns; and (4) Advanced lens designs—Diffractive optics can be manufactured using techniques like photolithography and electron-beam lithography, allowing for the creation of complex and precise optical structures that are impossible to achieve with traditional grinding and polishing methods.

These new systems are more economically viable because they address several key cost drivers and introduce novel efficiencies such as (1) elimination of inventory and waste. By adopting a just-in-time production model, manufacturers can do away with the vast, expensive inventory of various IOL powers and types. This eliminates storage costs, product expiration waste, and the logistical complexity of managing a large number of SKUs. (2) Reduced labor and capital expenditure. The entire process is highly automated. The specified software or Al handles the complex design phase, and the laser-based manufacturing cell requires minimal human intervention. This reduces the need for highly skilled technicians on the factory floor and lowers overall labor costs. The centralized design platform and standardized manufacturing cell also reduce the initial capital investment required compared to traditional, large-scale manufacturing plants. (3) Premium pricing for customization. This system allows for the creation of a truly premium, customized product. The ability to correct all HOAs offers patients a level of visual quality and spectacle independence that is currently unavailable with mass-produced lenses. This justifies a higher price point, making the entire operation profitable even at lower volumes. The high price is also offset by the potential for reduced post-operative adjustments, which are a hidden cost of traditional cataract surgery. (4) Enhanced post-surgical adjustment: A further application would be the potential for post-operative adjustment. Using the same laser or other light-based technology, minor adjustments to the IOL's optical properties could be made while the lens is in the eye, without surgery similar to the Light Adjusted Lens. One embodiment is that the laser could induce a chemical reaction in a targeted area of the IOL, which can alter the refractive index of the material. This technique could be used to adjust spherical power, correct astigmatism, or even add or remove multifocal features. This reduces the risk and cost of re-operation and ensures the patient receives the best possible visual outcome. These new methods and processes for manufacturing can ensure quality, mitigate operational risks, and position themselves as a clear leader in the high-growth, premium IOL market.

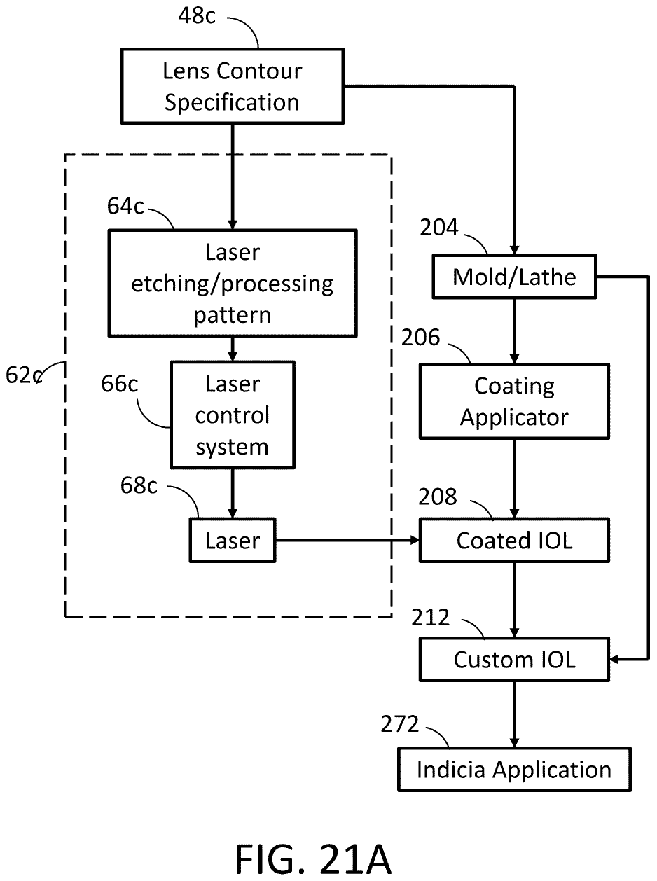

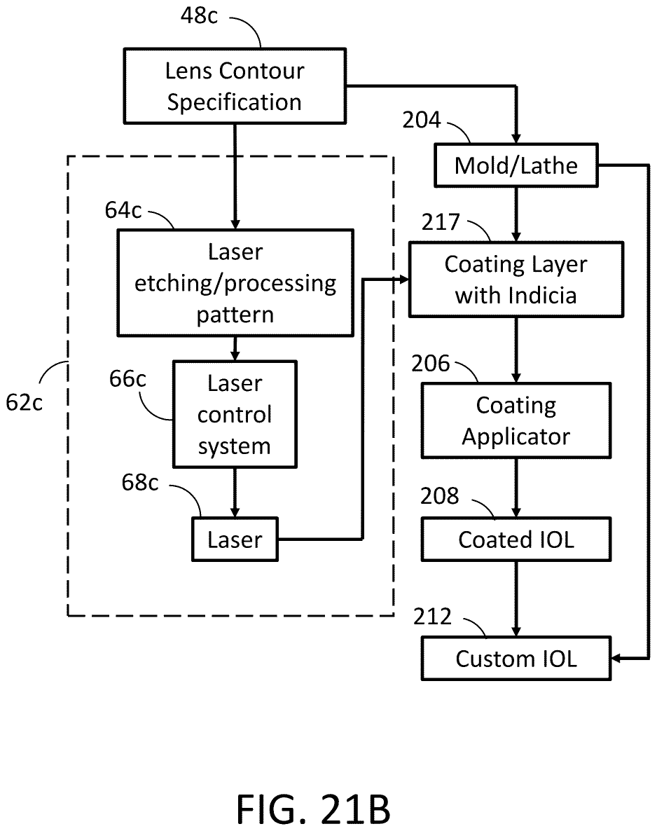

The systems of FIGS. 21A and 21B show some examples of the above-described systems and are analogous to those shown in FIGS. 14A and 14B, except instead of a lens for glasses or a contact lens being manufactured, it is an intraocular lens, preferably including corrections for HOA and the above-described indicia. As shown in FIG. 21A, lens contour specification 48c is generated, which may be used as an input to a laser system 62c portion and/or a mold/lathe 204 portion of a lens manufacturing process. A laser etching/processing pattern 64c derived from the lens contour specification 48c may be used by a laser control system 66c to control laser position, pulse duration, and the like for the laser 68c to emit the appropriate laser pulses in accordance with the lens contour specification 48c. As in the systems of FIGS. 14A and 14B, instead of performing ablation on a cornea, laser etching or other processing methods are performed to customize a lens for intraocular implantation to correct HOA of the wearer's eyes which may be determined by wavefront aberrometry or by corneal topography. In some embodiments, the selection of which to use may be performed using the methods and systems described herein. Astigmatism corrections incorporated into the intraocular lens may also be determined using the methods and systems described herein.

As shown in FIG. 21A, a lens may be processed by a mold/lathe 204 to generate the desired corrections for lower order aberrations myopia, hyperopia, and lower order astigmatism. These corrections may be derived from the lens contour specification 48c. Following molding or lathing, the lens is then processed by a coating applicator 206 which affixes a coating layer to the lens, which may be on the back side facing the retina in use. The coating layer may be designed thin enough and with an appropriate index of refraction to not significantly affect the optical performance of the lens as it exists prior to application of the coating layer. The coating layer of the coated lens 208 may then be laser processed with the laser 68c in accordance with the lens contour specification 48c to imprint corrections for the subject's HOA into the coating layer. The laser may etch the layer material with the HOA corrections. Another alternative is to use a layer material that is heat modifiable, where the imprinting of the HOA correction is done by selective heating and local deformation of the coating layer. The output of the process is a custom lens 212 which incorporates corrections for both lower order and higher order aberrations. In some embodiments, the molding/lathing performed at block 204 can implement both the lower order and higher order corrections defined by the lens contour specification, and the laser processing of a coating layer is not performed. Although not shown in FIG. 21A or 21B, it is also possible that the coating layer is processed mechanically, chemically, thermally, or by another means to incorporate the HOA corrections instead of by a laser as shown. Once the custom IOL is formed, a positioning indicia may be applied to the lens and/or haptic portions of the IOL at block 272 for use as described above. In some embodiments, the positioning indicia may be etched into the layer by the same laser treatment during the creation of the HOA corrections at block 208 (or block 217 of FIG. 21B) As shown in FIG. 21B, another option is to etch/process a coating layer as described above with HOA corrections before it is attached to a lens that has been processed to provide the sphere and astigmatism corrections. In this embodiment, the coating layer may already be provided with a positioning indicia prior to being processed to include HOA aberration corrections. Thus, with the systems of FIGS. 21A and 21B, intraocular lenses can be manufactured from the lens contour specifications generated with the above-described systems and methods which include custom corrections for HOA.

### Presbyopia Treatment

The systems and methods described herein can also be used to execute a novel laser ablation treatment for presbyopia. Presbyopia refers to the loss of near vision due from age related stiffening of the natural crystalline lens. This loss of flexibility prevents the muscles around the lens to flex the lens to change the focus for near work such as reading. As this lens system becomes stiff, it reaches a critical point in the early 40's and progresses where there is virtually no lens flexibility by a person's late 50's or early 60's. This is a problem every human being will contend with, unless they are naturally slightly nearsighted (which obviously compromises their distance vision. Thus, treatments for presbyopia are in high demand, and the search for an effective treatment is ongoing. Currently there are the following commercialized treatments:
Spectacles: reading glasses, bifocal glasses (these have an upper distance correction and a lower fixed reading correction, progressive glasses (these replace the lower fixed reading correction with a variable correction that increases in power as you move your gaze downwards. This has the advantage of added flexibility, but the disadvantage of a smaller working zone for a particular power. Many patients have problems with their vision due to the two or more zones in their glasses, especially with looking down as they are walking, missing steps, curbs etc, and many patients complain they make them dizzy and uncomfortable. Those patients will require completely separate glasses for distance and reading, and switch the two as necessary.

Monovision contact lenses: this technique corrects the dominant eye for distance vision, and the non-dominant eye for close or mid vision. There are limitations on how strong the reading vision can be to allow for neuro-adaptation so the brain can fuse the images and utilize the system, limitations with night glare and night vision because of the different eyes, and also limitations with patients who do not tolerate contacts or have corrections such as high astigmatism that soft contact lenses do not treat well.

Bifocal contact lenses: contacts with two or more circular zones with different powers. See multifocal intraocular lenses below.

Monovision laser vision correction: the same as above, except the correction is done on the cornea. This is better tolerated than contact lenses, as there is no problem with contact lens tolerance. It also creates a stable difference between the two eyes that the brain can neuro-adapt to, and also astigmatism correction is much better via laser than with contacts.

Corneal implants to induce corneal change or extended depth of focus (respectively Raindrop and KAMRA). Complications from corneal response to these as well as placement issues limited their effectiveness, and the side effects of the corneal reaction caused surgeons to stop using them.

Replacement of the natural crystalline lens (during cataract surgery) with an artificial lens with multiple circular focal zones or a lens with a progressive change to the power (similar to a progressive spectacle) allowing for reading power without causing the diffraction problems with multiple zones (Extended depth of focus or EDOF lens). An example is the Vivity intraocular lens by Alcon. These presbyopic treating IOLs have problems as well, as multifocal lenses will diffract light between the zones and patients can have significant night glare and visual issues. The EDOF lenses have the issue of not working if the pupil is not in the right position and the brain does not neuro-adapt to use these extended depth of focus zones.

Some other presbyopic corneal treatments have tried to make a centrally steeper cornea for monovision with some progressive affect but those treatments failed because the corneal steepening affect would decrease over time.

In summation of past treatments, they have either been corneal based (excimer laser, contact lens, and corneal implants), or intraocular lens based (replacing the natural crystalline lens usually when cataract surgery is needed), and they follow the same principles of either multiple zones of refraction, or an extended depth of focus strategy.

Attempts to find corneal refractive laser treatments for presbyopia that utilize either multifocal or EDOF strategies have met with limited success because of some of the same side effects as lenses, but also because the epithelium will compensate for a cornea that does not have a smooth, progressive curvature. The epithelium has been shown to compensate for irregularities in the cornea, and therefore the most successful excimer laser procedures have been with lasers that create smooth laser profiles that minimize epithelial compensation. Furthermore, epithelial compensation can also occur due to the natural higher order aberrations of the cornea. In treating only lower order aberrations such as lower order astigmatism and sphere (as in most LASIK), the change to the natural HOA could cause epithelial compensation that would also affect such corrections as a multifocal cornea or an extended depth of focus cornea.

Some of the failure or deficiencies in prior presbyopic laser correction treatments stems from the same problem that exists in overall laser correction treatments: the focus for treatments as been based on the existing medical and scientific basis that require an ablation pattern derived from “whole” eye measurements, rather than the Inventor's clinical studies that show that an ablation pattern should be based from other data necessary to create as uniform of cornea as possible, not a cornea that is ablated for theoretical posterior irregularities or ignores epithelial compensation effects on HOAs. The novel features of this invention begin with the Inventor's new scientific understanding that any procedure must create as uniform of cornea as possible. Therefore, the base cornea where a presbyopic laser correction treatment can be performed on would ideally be a cornea without significant higher order aberrations that could not only cause distortions in important areas of the extended depth of focus zone (EDOF zone) but could also induce epithelial compensation that would impact this EDOF zone. Therefore, the cornea must be as uniform as possible, which may be accomplished using the 032 patent systems and methodologies utilizing the LYRA™ Protocol to make a uniform cornea. Performing such a procedure on the cornea has advantages over an intraocular lens, as it would be performed on the first, anterior and therefore most important refracting surface. It would also be anterior to the iris, which can block intraocular peripheral EDOF zones.

In the process of making a more uniform cornea, the peripheral shape could be smoothly changed to create an EDOF circular zone to act in a similar way to a progressive spectacle lens which has a change in curvature to change the power to induce more myopia outwardly from the center 3 mm of the laser ablation pattern. This progressive change in curvature may be similar to that found in EDOF intraocular lenses. This curvature change is called the Q ratio, which defines the rate of curvature change across the human cornea. The natural cornea is not a dome, but aspheric. The average change of curvature from periphery to the center of a natural human cornea has a q ratio of 0.28. By manipulating the Q ratio and changing it across the peripheral cornea in an already uniform cornea, an extended depth of focus could be achieved that would not induce epithelial compensation and also would not scatter light with abrupt transition zones that would cause glare and night halos. This induced spherical aberration may cause some haloing around lights, but it likely would be a minimal trade-off for the enhanced reading as spherical aberration is well tolerated and the least disruptive to vision.

Embodiments of this procedure may comprise the following:
The creation of a uniform cornea, which has removed all HOA and corrected lower order astigmatism and sphere, and ablated a central region of the anterior portion of the cornea in accordance with a first Q-factor and ablated a peripheral region around the central region of the anterior portion of the cornea in accordance with a second, different Q-factor. This would be created from a map utilizing the following:
A. Topography measured HOA and lower order astigmatism and axis;
B. Wavefront and/or manifest measured refraction and total ocular astigmatism;
C. A software component that will compare steps A and B to determine if posterior ocular astigmatism exists and to treat accordingly;
D. OCT measured epithelial thickness mapping to provide a map of epithelial compensation of anterior stromal irregularity;
E. A software component that combines the resulting map from A, B, and C together with D to create a final comprehensive map of the cornea.
E. Measurement of the Q ratio from the corneal topography or other device that measures corneal curvature.
F. An ablation pattern that follows the pre-procedure natural q ratio for the central ablation area, likely to be 3 mm, but may be as small as 2 mm or even 1.5 mm.
G. An ablation pattern that then deviates from the central area to the periphery of the ocular ablation zone to provide extended depth of focus (EDOF), usually out to 6-7 mm. This may be referred to as the EDOF zone and may manipulate the Q ratio in a way similar to the defocus curves of EDOF intraocular lenses to allow for reading vision from the extended depth of focus.

A final part of the ablation zone may be from the outer edge of the EDOF zone to about 9 mm, or the transition zone that follows the final original transition zone based on the natural Q ratio or some other Q ratio that will be determined during testing. This is relatively unimportant as the vast majority of patients that are presbyopic have pupils smaller than 6.5 mm.

In summary, this invention treats presbyopia with an enhanced corneal laser ablation system and procedure that includes reshaping a cornea so that it is emmetropic (no correction) in the center region, with the peripheral zone decreasing in power more rapidly to create the myopia zone so that the peripheral zone is the reading area for the subject. Embodiments of this procedure may create a uniform cornea that would be derived from utilizing topography information and/or wavefront and/or Scheimpflug camera information combined with OCT epithelial mapping irregularity, but would also change the Q ratio outside the central ablation area (likely 1.5-3 mm) of correction to create a circular EDOF zone that would be similar to a progressive pair of glasses or a EDOF intraocular lens. This new medical procedure by the Inventor is named the Q-RESHAPED™ Protocol (Q-Ratio Enhanced Shape Healing Adjusted Presbyopia Extended Depth Protocol). The system and methods to perform this new breakthrough medical procedure are embodiments of this invention.

The Q-RESHAPED™ Protocol has two fundamental differences from past proposed treatments. First, it focuses on creating a uniform cornea, specifically treating HOAs and epithelium compensation, which past treatments essentially ignored. Second, it treats presbyopia by reshaping a cornea so that it is emmetropic (no correction) in the center region, with the peripheral zone decreasing in power more rapidly to create the myopia zone so that the peripheral zone is the reading area for the subject; whereas, prior treatments attempted to make the central zone the reading area and decrease the induced myopia peripherally-just the opposite of this invention. Prior proposed treatments essentially create an aberrant cornea that will have the epithelium compensate for the aberration and throw the ocular focusing system off.

Epithelium compensation occurs least on a smooth gradual surface. By doing the opposite of prior proposed procedures, we create a smoother gradation, for example using a Q Ratio of 0.8 rather than the natural 0.28. This will prevent epithelial compensation from occurring and filling in the area and ruining the corrective effect.

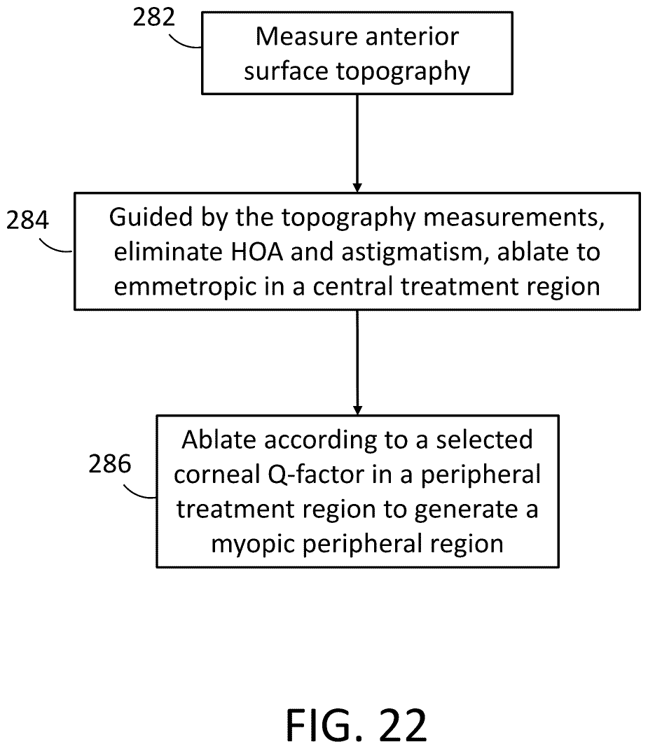

FIG. 22 is a flowchart illustrating the ablation principles described above for treating presbyopia. Referring now to FIG. 22, the method starts at block 282 by measuring the anterior surface topography. With this topographic information generated at 282, ablation is performed to eliminate HOA and astigmatic topographical features of the anterior cornea to produce a smooth substantially emmetropic anterior corneal surface in a central region at block 284. The central emmetropic region to which this topographic guided ablation is applied may be 1.5 to 3 mm in diameter.

At block 286, a peripheral region referred to herein as the extended depth of focus (EDOF) region is created. The EDOF region may extend from the perimeter of the central emmetropic region out to about 6-7 mm diameter. In this EDOF region, Q-factor guided ablation may be used to create a myopic peripheral region. With this ablation treatment for presbyopia, the central region is used for distance vision, and the peripheral myopic region is used for reading.

There are several advantages to this presbyopia treatment method. First, when reading, the visual axis tends to shift downward away from the corneal apex. Because the myopic region used for reading is in the peripheral region rather than the central region, the visual axis will often shift into this myopic region when the subject wants to read. In addition, using topographic guided ablation in the central region and Q-factor guided ablation in the peripheral region helps minimize aberrations at the transition between the central and peripheral regions, which minimizes visual aftereffects created by such aberrations and also minimizes post treatment epithelial compensation that will reduce the myopic curvature introduced by the ablation in the peripheral region.

Although epithelial thickness mapping to provide a map of epithelial compensation of anterior stromal irregularity is considered an important feature of the Q-RESHAPED™ procedure, embodiments of this procedure may include determining epithelial compensation from methodologies other than using an OCT device, such as algorithms to determine theoretical epithelial compensation based on other measuring techniques.

Further, as an alternative to epithelial mapping, embodiments include configuring a wavefront and/or topographic guided ablation system, without OCT, as follows:

Measuring the Q ratio from corneal topography and/or wavefront.

Determining an ablation pattern that follows the pre-procedure natural q ratio for the central ablation area, likely to be 3 mm, but may be as small as 2 mm or even 1.5 mm.

Determining an ablation pattern that then deviates from the central area to the periphery of the ocular ablation zone to provide extended depth of focus (EDOF), usually out to 6-7 mm. This may manipulate the Q ratio in a way similar to the defocus curves of EDOF intraocular lenses to allow for reading vision from the extended depth of focus.

A final part of the ablation zone may be from the outer edge of the EDOF zone to about 9 mm, or the transition zone that follows the final original transition zone based on the natural Q ratio or some other Q ratio that will be determined during testing.

Embodiments of this invention include corneal laser ablation systems configured and programmed to perform the methodologies derived from the disclosures herein. Another option that may be provided in the menu embodiment of FIG. 10B is an option to select a target Q-factor for the anterior cornea following the ablation treatment. With this option, a selected Q-factor can be another guide for ablation map generation.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

1. An intraocular lens comprising:
an optic portion; and
a haptic portion;
wherein the optic portion comprises corrections for higher order aberrations, wherein the corrections are derived at least in part from an aberration map of an anterior corneal surface of a subject, and wherein the corrections comprise corrections for one or more 3^{rd} or 4^{th} order corneal higher order aberrations selected from trefoil, coma, quadrafoil, and higher order astigmatism that are present in the aberration map.

2. The intraocular lens of claim 1, wherein the corrections are derived at least in part from wavefront aberrometry data, topography data, and/or optical coherence tomography (OCT) data.

3. The intraocular lens of claim 1, wherein the corrections for higher order aberrations are incorporated into a coating layer applied to the optic portion or are incorporated into the optic material itself.

4. The intraocular lens of claim 1, wherein the optic portion comprises an extended depth of focus profile for presbyopia correction.

5. The intraocular lens of claim 1, wherein the intraocular lens comprises positioning indicia.

6. The intraocular lens of claim 5, wherein the positioning indicia are formed by laser etching.

7. The intraocular lens of claim 6, wherein the positioning indicia are located on both the optic portion and the haptic portion.

8. The intraocular lens of claim 1, 7, wherein the aberration map is obtained at least in part by using Placido ring imaging or Scheimpflug imaging.

9. The intraocular lens of claim 1, wherein the corrections for higher order aberrations are incorporated into the optic portion by laser etching, molding, lathing, adaptive optics, diffractive optics, or through 3D printing production.

10. The intraocular lens of claim 9, wherein the corrections comprise a mirror image of the anterior corneal surface defined by the aberration map to refractively cancel higher and lower order aberrations.

11-36. (canceled)

37. A system for surgically implanting an intraocular lens comprising:
a surgical viewer;
an eye tracker; and
a laser that is coordinated with the eye tracker and configured to inject a laser beam into an intraoperative optical viewing path to create a laser spot on the cornea while implanting the intraocular lens;
wherein the laser is configured to receive information regarding a desired location of a portion of an intraocular lens with respect to an anterior corneal surface of a surgical subject; and
wherein the desired location aligns higher order aberration corrections in the intraocular lens with higher order aberrations present on the surface of the cornea.

38. The system of claim 37, wherein the location of a portion of an intraocular lens comprises a positioning indicia marked on the intraocular lens.

39. The system of claim 37, wherein the surgical viewer comprises a digital image acquisition device.

40. The system of claim 37, further comprising a robotic arm configured to push or otherwise manipulate the intraocular lens during the surgery either manually or by computer processes.

41. The system of claim 40, wherein the robotic arm is configured to align a positioning indicia on the intraocular lens with the laser spot.

42-45. (canceled)

46. A system for surgically implanting an intraocular lens into an eye of a subject, the system comprising:
a surgical viewer; and
a wavefront aberrometer, wherein the wavefront aberrometer comprises a display configured to display optical aberrations of the eye of the subject during a surgical procedure while the intraocular lens is manipulated during and after the implanting.

47-53. (canceled)
